SEQUENCE LISTING (370 sequences)

SEQ ID NO: 1 - DNA, 20 bp, Artificial Sequence
  Antisense Compound
tccgtcatcgctcctcaggg

SEQ ID NO: 2 - DNA, 20 bp, Artificial Sequence
  Antisense Compound
gtgcgcgcgagcccgaaatc

SEQ ID NO: 3 - DNA, 20 bp, Artificial Sequence
  Antisense Compound
atgcattctgcccccaagga

SEQ ID NO: 4 - DNA, 4266 bp, H. sapiens
  CDS (10)...(1224)
tggggtgacatgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtagcagaacttcacccccttttaagctacagttccttctctccatccttcgaccttcacaaaatctctgggactgttctttgtcagattcttcctcctttagaaggctgggtcccattctgtccttcttgtcatgcctccaatttcccctggtgtagagcttgtttttctggcccatccttggagctttatgagtgagctggtgtgggatgcctttgggggtggacttgtgttccaagaatccactctctcttccttttggagattaggatatttgggttgccatgtgtagctgctatgtcccctggggcgttatctcatacatgcaaacctaccatctgttcaacttccacctaccacctcctgcacccctttgatcggggacttactggttgcaagagctcattttgcaggctggaagcaccagggaattaattcccccagtctaccaatggcacccagagagggcatggaggctccacgcaaccccttccacccccacatcttcctttgtcttatacatggcttccatttggctgtttctaagttgtattctttattttattattattattactatttttcgagatggagtttcactcttgtcgctcaggctggagtgccatggcgcgatcttggctcactgcaacctctgcctcccgggttcaagtgattctcctgcctcagcctcacgagtagctggaattacaggcaggcgccaccaggcccggctaattttttgtatttttagtacagacggggtttctccgtgttggtcaggctggtcttgaactcccgacctcagatgatctgcccgcctcggcctcccaaaattgctgggattacaggtgtgagccaccgcgcctggcctattattttttgtaagaataaaacaggtttactgggatttgggactctgaacagttctgtctctactacctgatctcctcctaccacgactttgggatctagaggagctttggctccggctgtgacggctccggccgttctcactgcggctgcaccggcccccgctgcggtcactatttcttcctctgcttggtgaattgtgcctctcctggctctttgacatgtgctagtgagatttcttccttttcctttcggattccccatttcttttgtaggaatggtctggactagggttctccttccccgcagcctgtagtattcatcgtggtggcccatcctctctctccccttggagctcttgccaaaggaggagacaagcagaggtctctattggatttctcaacacctgaagaaagttgcagtgttttcctcttggacattgttgtatttcaaataaaccacaaatcatcattttccaccgagccactgggcagaattcacactgaagctgtcgtcctgcgtacataccatcgtccgttaaacagagaaagagctgcttggcattcttcttccgactggtactgaacatatatacttgcccctcaggtgaggttccaagttgcaactgaccttgaactgaatcactctccccacgttattttttaattactatttttttttaaagatggggtcttgctctgtcgccaggctggagtgcagtggcgcgatctaggctcactgcaacttccgcctcccgggttcaagcgattctcctgcctcagcctcccgagtagctgggactctactaaaagtacaaaaattagctggcgtgcaccaccgcgcccagctaattcttgtatttttggtagagacggggtttcaacatgttgaccaggatggtctcgatctcttgacctcgtgattcgcccgccgcgtcctcccaaagtgctgggattacaggcctgagccaccgcgcccagtctctccccacgttcttgaactcgggcagcacatcctcacagaaatctaggaactgttggtaggtttcttcctcgctgtactccaggcttgcttcggagtcatagtcatccctcctgcactgctcctttccaaacactgtaaacatgcttttaataagaagggtaggactggatgttgggaaatcatgtgaacatctatctccaaatctgcaagctcctgttttactgtagaagggacaattaactccatccttctccatgactctgaaatccaagggggggttccgggttttgccatgtggcgccattttccaactcattttcagcctgatccagcatcttctggacagcttccggtttttgtttcttctgtcgtttctgttcctcctcctctctctctttcctctgctgttcttcccattgttcctttaactttcgctcttgttcttgccgttttctagccacctcttccttttccttctttattctgaattcttcttgtgccttctgctctctcagcaaccactcctcatgtaatctttgcctctctcttccccatagcttttctagttgttgtttttcaataaaagtgtcctcctctttctgtgagagtcctgagtccctcagtggagcaagttcctgctggcgtttctttcgtttctccttcttcagggcggccctgtactttttgtggcttggtttctctggaaatgtcaccttttcgggcgcagccatcttgccggcaccgccccgcccctctagttgtatcctttataataaaatggtaaacattgtaaccgcagattcagcccaatctggttcaactttgtgtaataaaatggcgagttgtttttcagttgtcgtggacccccaggttgcaagttacataccctgggcatgtccagatgaacgaagcgtgcaaatccacgtggaacctaagtgctcagactgaggaacagggactgagttaagaagtggacaccacgtggcatgatccttgatccaatcagattgagccctggcgtgatccagtcagatcaagcctcctgaatcccctcattacaagatccaatcatatcatgcctcactaccctctgtatataaaatctgccccagcctccaacttggagagacagatttgggccagactcctgtgtccttgcttggctgccttgcaataaatttttctctctaca

SEQ ID NO: 5 - DNA, 28 bp, Artificial Sequence
  PCR Primer
catgcctcactaccctctgtatataaaa

SEQ ID NO: 6 - DNA, 20 bp, Artificial Sequence
  PCR Primer
acacaggagtctggcccaaa

SEQ ID NO: 7 - DNA, 26 bp, Artificial Sequence
  PCR Probe
ccccagcctccaacttggagagacag

SEQ ID NO: 8 - DNA, 19 bp, Artificial Sequence
  PCR Primer
gaaggtgaaggtcggagtc

SEQ ID NO: 9 - DNA, 20 bp, Artificial Sequence
  PCR Primer
gaagatggtgatgggatttc

SEQ ID NO: 10 - DNA, 20 bp, Artificial Sequence
  PCR Probe
caagcttcccgttctcagcc

SEQ ID NO: 11 - DNA, 1484 bp, M. musculus
  CDS (79)...(795)
ctaagtaagattcaaggaggtggcaaataaaagctgtagtctctgtcagattctggcactgagaagtggctgtgaaacatgagtgattctaaggaaatggggaagaggcagcttcgccctctggatgaggaactgctgacatccagccacaccaggcactccatcaaaggctttggcttccaaacaaattctggattcagtagcttcacagggtgcctggtccacagtcaagtccccttggcactgcaggtgctcttcctagctgtttgttctgtgctgctggttgtcatccttgtcaaagtctacaaaatacccagttctcaggaagaaaacaatcagatgaatgtctaccaagaactgacccagttgaaggctggcgtagatcgactgtgccgctcctgcccctgggactggacgcacttccaaggaagctgttacttcttctctgtggcccagaagtcctggaatgattctgccactgcctgccacaatgtgggggctcaacttgtggtcatcaagagtgatgaagagcagaactttctacaacaaacttctaagaagagaggctacacttggatggggctcattgacatgagcaaggagtctacatggtactgggtagatggttcacctctgactctcagtttcatgaagtattggagtaaaggagaacctaacaacctgggagaggaagactgtgcagagttcagagatgacggctggaatgacaccaaatgtactaacaagaaattctggatctgcaaaaagctttcaacttcctgccctagcaagtgatggccaactccctccaccatctccacagtcccaaaaccctgccaaatggcagaactttacccatagctatgccagtttattctacttgtctgtgaccattgataaccttgacaagatctgtaagactttattctggcaggtagcttgtctcctgcattgctcctaggtccagtttgtctcctggaccattaatgtatagtctcctgatgtctgcaggttctctttgggttagtggttgtggctctggtccacctttgacatttaatgaagtaaacagacatgaggtgtccttaaatgggttcctttcatctcctctgtcttcctttctgagtagagaccacatattgccatgtgcaattgatcctgagtactcacacctactaaattttaattcatctaccctgcacacctttctatgggacccttctctgatttcaggagctcaacctatagactaaaagcacaagagaaccaattccttaactattagtcatgatcagaaattattgtgttaattctctgactccatctctacccctgggtggacatggtgagcctcaatgttatagacctcccaaattttttggaggatttataatttttattgaacttactcattaacaattgtatacatatgtatgtataaatataagtataaataaacattctgatttttgccatccttcatc

SEQ ID NO: 12 - DNA, 21 bp, Artificial Sequence
  PCR Primer
cctctggatgaggaactgctg

SEQ ID NO: 13 - DNA, 19 bp, Artificial Sequence
  PCR Primer
tggaagccaaagcctttga

SEQ ID NO: 14 - DNA, 23 bp, Artificial Sequence
  PCR Probe
catccagccacaccaggcactcc

SEQ ID NO: 15 - DNA, 20 bp, Artificial Sequence
  PCR Primer
ggcaaattcaacggcacagt

SEQ ID NO: 16 - DNA, 20 bp, Artificial Sequence
  PCR Primer
gggtctcgctcctggaagat

SEQ ID NO: 17 - DNA, 27 bp, Artificial Sequence
  PCR Probe
aaggccgagaatgggaagcttgtcatc

SEQ ID NO: 18 - DNA, 1312 bp, H. sapiens
ctaaagcaggagttctggacactgggggagagtggggtgacatgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtagcagaacttcacccccttttaagctacagttccttctctccatccttcgacctttag

SEQ ID NO: 19 - DNA, 1197 bp, H. sapiens
  CDS (1)...(1197)
atgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 20 - DNA, 939 bp, H. sapiens
  CDS (1)...(939)
atgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 21 - DNA, 507 bp, H. sapiens
  CDS (1)...(507)
atgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 22 - DNA, 1143 bp, H. sapiens
  CDS (1)...(1143)
atgagtgactccaaggaaccaagactgcagcagctgggcctcctggaggaggaacagctgagaggccttggattccgacagactcgaggatacaagagcttagcagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 23 - DNA, 1083 bp, H. sapiens
  CDS (1)...(1083)
atgagtgactccaaggaaccaagactgcagcagctgggcctcctggtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 24 - DNA, 1315 bp, H. sapiens
  CDS (101)...(1315)
atgagtgactccaaggaaccaagactgcagcagctgggcctcctgggtgaggctgggttgggacgctgggattctgggaagggggaagggatggccagccatggcctcagcctgcccaggctctgatttcacgtctatccattcagaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 25 - DNA, 994 bp, H. sapiens
  CDS (101)...(994)
atgagtgactccaaggaaccaagactgcagcagctgggcctcctgggtgaggctgggttgggacgctgggattctgggaagggggaagggatggccagccatggcctcagcctgcccaggctctgatttcacgtctatccattcagaggaggaacagctgagaggccttggattccgacagactcgaggatacaagagcttagcagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagcttcaagcagtattggaacagaggagagcccaacaacgttggggaggaagactgcgcggaatttag

SEQ ID NO: 26 - DNA, 2328 bp, H. sapiens
ttttcgagatggagtttcactcttgtcgctcaggctggagtgccatggcgcgatcttggctcactgcaacctctgcctcccgggttcaagtggttctcctgcctcagcctcacgagtagctggaattacaggcaggcgccaccagacccggctaattttttgtatttttagtacagatggggtttctccgtgttggtcaggctggtcttgaactcccgacctcagatgatctgcccgcctcggcctcccaaaattgctgggattacaggtgtgagccaccgcgcctggcctattatttttttgtaagaataaaacaggtttactgggatttgggactctgaacagttctgtctctactacctgatctcctcctaccacgactttgggatctagaggagctttggctccggctgtgacggctccggccgttctcactgcggctgcaccggcccccgctgcggtcactatttcttcctctgcttggtgaattgtgcctctcctggctctttgacatgtgctagtgagatttcttccttttcctttcggattccccatttcttttgtaggaatgatctggactagggttctccttccccgcagcctgtagtattcatcgtggtggcccatcctctctctccccttggagctcttgccaaaggaggagacaagcagaggtctctattggatttctcaacacctgaagaaagttgcagtgttttcctcttggacattgttgtatttcaaataaaccacaaatcatcattttccaccgagccactgggcagaattcacactgaagctgtcgtcctgcgtacataccatcgtccgttaaacagagaaagagctgcttggcattcttcttccgactggtactgaacatatatacttgcccctcaggtgaggttccaagttgcaactgaccttgaactgaatcactctccccacgttattttttaattactatttttttttaaagatggggtcttgctctgtcgccaggctggagtgcagtggcgcgatctaggctcactgcaacttccgcctcccgggttcaagcgattctcctgcctcagcctcccgagtagctgggactctactaaaagtacaaaaattagctggcgtgcaccaccgcgcccagctaattcttgtatttttggtagagacggggtttcaacatgttgaccaggatggtctcgatctcttgacctcgtgattcgcccgccgcgtcctcccaaagtgctgggattacaggcctgagccaccgcgcccagtctctccccacgttcttgaactcgggcagcacatcctcacagaaatcatgtgaacatctatctccaaatctgcaagctcctgttttactgtagaagggacaattaactccatccttctccatgactctgaaatccaagggggggttccgggttttgccatgtggcgccattttccaactcattttcagcctgatccagcatcttctggacagcttccggtttttgtttcttctgtcgtttctgttcctcctcctctctctctttcctctgctgttcttcccattgttcctttaactttcgctcttgttcttgccgttttctagccacctcttccttttccttctttattctgaattcttcttgtgccttctgctctctcagcaaccactcctcatgtaatctttgcctctctcttccccatagcttttctagttgttgtttttcaataaaagtgtcctcctctttctgtgagagtcctgagtccctcagtggagcaagttcctgctggcgtttctttcgtttctccttcttcagggcggccctgtactttttgtggcttggtttctctggaaatgtcaccttttcgggcgcagccatcttgccggcaccgccccgcccctctagttgtatcctttataataaactggtaaacattgtaaccgcagattcagcccaatctggttcaactttgtgtaataaaatggcgagttgtttttcagttgtcgtggacccccaggttgcaagttacataccctgggcatgtccagatgaacgaagcgtgcaaatccacgtggaacctaagtgctcagactgaggaacagggactgagttaagaagtggacaccacgtggcatgatccttgatccaatcagattgagccctggcgtgatccagtcagatcaagcctcctgaatcccctcattacaagatccaatcatatcatgcctcactaccctctgtatataaaatctgccccagcctccaacttggagagacagatttgggccagactcctgtgtccttgcttggctgccttgcaataaatttttctctctac

SEQ ID NO: 27 - DNA, 8351 bp, H. sapiens
agcaggaattcccaaaaatgaggacagcagcagctcaaagcaggaaagccaggaggtcacaggggacagtgcttccaggaactgggggtgctacctgcctacccttgccctagtggagggggtgtaacacagtagctttctcttacaatttatcagatttcattaaaagcatcccactgctcagccatccatgagtggatgagtccttctccctgtcaaccccagaccatcccccactgcctccctgaaattcttgaaagatccggcccatctcatgctctgatgctttccactagtcttttggatgacagatccctacccaacttcctgtttctctttctgtgggagactagatttaggaagtaaagatcacagggtgggaaataaaagctgtggcccccaggagttctggacactgggggagagtggggtgacatgagtgactccaaggaaccaagactgcagcagctgggcctcctgggtgaggctgggttgggacgctgggattctgggaagggggaagggatggccagccatggcctcagcctgcccaggctctgatttcacgtctatccattcagaggaggaacagctgagaggccttggattccgacagactcgaggatacaagagcttagcaggtagagaggacttagcagtttgggaggagagacgagagactcctggactcctgggtcctgaggagctggacgctggggcctggactcttgggtcctggggatgtggggctaggggcctggactcctgggacctgaggaggttgagtttggggcctgaattcctggttcctgggaaggtggaggctggggccaagactcctgggttctggggaggtagaagttgaagcctgggttcctgggtcctggggaggtggggtcaggggcctagactcctgggtcctggagaggtgggggcctgggcctggactcctgggtcatggggaggtggggacttgggcctgggttcctgggacctggggatgtaggggctgggacccagattcttgggtcttggggaggtgggagcaggtacctgggttactgggacctggggaaggggaggctgggtcctgggtccctgggtcctggggaggtgggagctgggggcctgggttccagggacctgggaatgtagaggctggggcctggattcctgggtcctggggaggtggaagcaggtacctggttcctgggacctggggaaggggaggctggggacggggtccctgggtcctggggaggttagagctagggcctggattcccaggtgttggagtggagggggctggggctggggttcctgggtcctggggtgctgagggatttagaccaggctctccctgggccaggctccggtggggacactggcaggctgacgcatgtatcctctctcagggtgtcttggccatggtcccctggtgctgcaactcctctccttcacgctcttggctgggctccttgtccaaggtcaggggcaggttctgagggtctggggtccccacgcctggcttttggctgtggacacagcctggggctgccagatctgggagggaggtgggactgagagccaagatgttgtccctgggggtctgtaggggcctctcccacagcccctccccactctagggcaggacaggagagggaggaggaggaggacaggaagaatctggctctccttggccttctgtgctgccttctccaggtgtcagttttcttcatcttcaaaggggattaaccaagaccttggctctcacaaatgatgtccctatggggctcagttcaaggcttggcacacagtaggtgtttaataattgcagttccttttttttcttggcccagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtgggtgagtatctggaaccagggcttcttgggcctgagatggtcgttgtgtggtgtgagtttgcttgagtcactgaccctgcctgagcaagtttcctcatctgtgaaatgagaacacagggtaactgtgatcattgccctcagtgttcacagtgggccaggtacacaacaggcactcagttaagccagtctcctgccttctgttctgagttctaaagccccaacttctcctagtctggaggagatggggccaggactcctgggttctcatggggatgggggagggtctcgggaagtggggaggggaatggtctacccggcgtggacagtcaaggaaggactctgactctcaagaccttgaaggctgggcaggtacaggaatatggacaacctaatctaagcccagccctgaccagcctcccccaacagaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcaggtacacctggtgggggtccccgtcctggcctagggcatggcttctggccaactggggaggaggtgctcagagatgactttgcagaagaggggttttgctccacttggaaagaagggaggagaaggatatgaatgagggagtctcaagtacccttagagcagatacaaaccaaatctgaacatgtgtgaatgctccggaaaaaaacatcaaatgtaagcaaacccctgcagcatgctgcaagaggttaacaaccagctctctggaggaagaagtcctgatgggtcgtgtttgccagtttccctggtgtaaatattcccatggtggctgatttcaagctgtcgttgtgatgtcactcagtgcagagctgaaaggagatgtgcatgaccagcagttaggaaatggttgcctctggctccaacacattaagcccagttatgagtaaatgtgcatggacatgcacagaacatgtgaaatggataaacatgcagtattagcaagagggtgtgaggagaggccagctgtgcatggatgtgtgcatgtcaggacacatgtgccgtgggatacacatgtgagagggactcagacacgagggggctcaggacctagtataccagaaggctgtaggttagcctacaagcagaacttcctaccaagagatggggagacagagcctggaaaatgggaagcagagctccctcctgactcctcctgtaccctccagaacttcctacagctgcagtcttccagaagtaaccgcttcacctggatgggactttcagatctaaatcaggaaggcacgtggcaatgggtggacggctcacctctgttgcccaggtagatctcagaaggggcaccctatccactggggctggaattgtgaaactctgatttgaactttccctggggatcctccccaactccctgacaccctagacccaagaatctggaggttttactgagttgaggaaggtggtcttcatacccacttccagacagcatggcaggtgcctagaattccaccagcagagctgcaggaggagcctgccaccggggaggtgtattaaaatataatgtattaataggaagtctgaggtccagtaaggccaccagctcaggagacacctgccattgtgaatagcttctcatatacacagatccctagagagaggacacatcacagctgggggccacctggggaagcaccagggtctgttatgagtcagagtggtggggaggggggactgtgggccagagcctttattatggtttctgtggaaaggaaaggtcaaggccaggtgaacaggcttaggattgggaagtgtgaagcattttttgggctctggggcacaggggctgcccctcgctgtctgacacctggccttggggtgattagtacaggatagtggcttggagtacaagagcccagtaaggcaggtcgctggaggtgtggactccagactggttggcttgtatttgaaaatcacacactcaataggtggagtccacccaaggacaggttgtgagaagagaggcaggaactcaaggcatcgtgagtcaggcatattatcaggtatgcagagggattcaagtccagcatacacacgcagggcagatgttaaggcatcaagatgacagaaggtagaaacacagctcatgtagctggcggggacagaggctcacattccgcatgggctttgcagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtagcagaacttcacccccttttaagctacagttccttctctccatccttcgaccttcacaaaatctctgggactgttctttgtcagattcttcctcctttagaaggctgggtcccattctgtccttcttgtcatgcctccaatttcccctggtgtagagcttgtttttctggcccatccttggagctttatgagtgagctggtgtgggatgcctttgggggtggacttgtgttccaagaatccactctctcttccttttggagattaggatatttgggttgccatgtgtagctgctatgtcccctggggcgttatcttatacatgcaaacctaccatctgttcaacttccacctaccacctcctgcacccctttgatcggggacttactggttgcaagagctcattttgcaggctggaagcaccagggaattaattcccccagtcaaccaatggcacccagagagggcatggaggctccacgcaaccccttccacccccacatcttcctttgtcttatacatggcttccatttggctgtttctaagttgtattctttattttattattattattactatttttcgagatggagtttcactcttgtcgctcaggctggagtgccatggcgcgatcttggctcactgcaacctctgcctcccgggttcaagtgattctcctgcctcagcctcacgagtagctggaattacaggcaggcgccaccagacccggctaattttttgtatttttagtacagatggggtttctccgtgttggtcaggctggtcttgaactcccgacctcagatgatctgcccgcctcggcctcccaaaattgctgggattacaggtgtgagccaccgcgcctggcctattattttttgtaagaataaaacaggtttattgggatttgggactctgaacagttctgtctctactacctgatctcctcctaccacgactttgggatctagaggagctttggctccggctgtgacggctccggccgttctcactgcggctgcaccggcccccgctgcggtcactatttcttcctctgctaggtgaattgtgcctctcctggctctttgacatgtgctagtgagatttcttccttttcctttcggattccccatttcttttgtaggaatggtctggactagggttctccttccccgcagcctgtagtattcatcgtggtggcccaccctctctctccccttggagctcttgccaaaggaggagacaagcagaggtctctattggatttctcaacacctgaagaaagttgcagtgttttcctcttggacattgttgtatttcaaataaaccacaaatcatcattttccaccgagccactgggcagaattcacactgaagctgtcgtcctgcgtacataccatcgtccgttaaacagagaaagagctgcttggcattcttcttccgactggtactgaacatatatacttgcccctcaggtgaggttccaagttgcaactgaccttgaactgaatcactctccccacgttattttttaattactatttttttttaaagatggggtcttgctctgtcgccaggctggagtgcagtggcgcgatctaggctcactgcaacttccgcctcccgggttcaagcgattctcctgcctcagcctcccgagtagctgggactccactaaaagtacaaaaattagctgggcgtgcaccactgcgcccagctaattcttgtatttttggtagagacggggtttcaacatgttgaccaggatggtctcgatctcttgacctcgtgattcgcccgccgcgtcctcccaaagtgctgggattacaggcctgagccaccgcgcccagtctctccccacgttcttgaactcgggcagcacatcctcacagaaatctaggaactgttggtaggtttcttcctcgctgtactccaggcttgcttcggagtcatagtcatccctcctgcactgctcctttccaaacactgtaaacatgcttttaataagaagggtaggactggatgttgggaaatcatgtgaacatctatctccaaatctgcaagctcctgttttactgtagaagggacaattaactccatccttctccatgactctgaaatccaagggggggttccgggttttgccatgtggcgccattttccaactcattttcagcctgatccagcatcttctggacagcttccggtttttgtttcttctgtcgtttctgttcctcctcctctctctctttcctctgctgttcttcccattgttcctttaactttcgctcttgttcttgccgttttctagccacctcttccttttccttctttattctgaattcttcttgtgccttctgctctctcagcaaccactcctcatgtaatctttgcctctctcttccccatagcttttctagttgttgtttttcaataaaagtgtcctcctctttctgtgagagtcctgagtccctcagtggagcaagttcctgctggcgtttctttcgtttctccttcttcagggcggccctgtactttttgtggcttggtttctctggaaatgtcaccttttcgggcgcagccatcttgccggcaccgccccgcccctctagttgtatcctttataataaactggtaaacattgtaaccgcagattcagcccaatctggttcaactttgtgtaataaaatggcgagttgtttttcagttgtcgtggacccccaggttgcaagttacataccctgggcatgtccagatgaacgaagcgtgcaaatccacgtggaacctaagtgctcagaccgaggaacagggactgagttaagaagtggacaccacgtggcatgatccttgatccaatcagattgagccctggcgtgatccagtcagatcaagcctcctgaatcccctcattacaagatccaatcatatcatgcctcactaccctctgtatataaaatctgccccagcctccaacttggagagacagatttgggccagactcctgtgtccttgcttggctgccttgcaataaatttttctctctacaaaaccccagtgcttcagtgtttggttttccactgtgagccagggaactgacccaatttagttcggcaacaacataagcaaaatgttttcccgagttctctaaatcattctaggaaattactgaacttgagatggacttatgggaagccacaattttttgccagcctggcagaagtgtgggtagacagtgcaaagactgtctttgcacctggcatctgaagcagggtcagtcttttgggacctagccctttaatttgtggaatgtggtgctaactccaggaagctagtgttagaattagagaatttctttggtgtaaataagacaccacaaacatcacttcctttctactgatgttgaccccaaagatgcaccttcataaacagcttgcaagctaaattcc

SEQ ID NO: 28 - DNA, 20 bp, Artificial Sequence
  Antisense Compound
tcccccagtgtccagaactc

SEQ ID NO: 29 - DNA, 20 bp, Artificial Sequence
  Antisense Compound
gagtcactcatgtcacccca

SEQ ID NO: 30 - DNA, 20 bp, Artificial Sequence
  Antisense Compound
gctgttcctcctccaggagg

SEQ ID NO: 31 - DNA, 20 bp, Artificial Sequence
  Antisense Compound
tcgagtctgtcggaatccaa

SEQ ID NO: 32 - DNA, 20 bp, Artificial Sequence
  Antisense Compound
caccctgctaagctcttgta

SEQ ID NO: 33 - DNA, 20 bp, Artificial Sequence
  Antisense Compound
tggacaaggagcccagccaa

SEQ ID NO: 34 - DNA, 20 bp, Artificial Sequence
  Antisense Compound
tcttagatttctctggaagc

SEQ ID NO: 35 - DNA, 20 bp, Artificial Sequence
  Antisense Compound
tgcttgaagctgggcaacag

SEQ ID NO: 36 - DNA, 20 bp, Artificial Sequence
  Antisense Compound
aatactgcttgaagctgggc

SEQ ID NO: 37 - DNA, 20 bp, Artificial Sequence
  Antisense Compound
atccagaatttggcaagatt

SEQ ID NO: 38 - DNA, 20 bp, Artificial Sequence
  Antisense Compound
gaaggaactgtagcttaaaa

SEQ ID NO: 39 - DNA, 20 bp, Artificial Sequence
  Antisense Compound
aatctgacaaagaacagtcc

SEQ ID NO: 40 - DNA, 20 bp, Artificial Sequence
  Antisense Compound
cttctaaaggaggaagaatc

SEQ ID NO: 41 - DNA, 20 bp, Artificial Sequence
  Antisense Compound
ggacccagccttctaaagga

SEQ ID NO: 42 - DNA, 20 bp, Artificial Sequence
  Antisense Compound
tcttggaacacaagtccacc

SEQ ID NO: 43 - DNA, 20 bp, Artificial Sequence
  Antisense Compound
ggcaacccaaatatcctaat

SEQ ID NO: 44 - DNA, 20 bp, Artificial Sequence
  Antisense Compound
gtaagtccccgatcaaaggg

SEQ ID NO: 45 - DNA, 20 bp, Artificial Sequence
  Antisense Compound
cctctagatcccaaagtcgt

SEQ ID NO: 46 - DNA, 20 bp, Artificial Sequence
  Antisense Compound
gccaaagctcctctagatcc

SEQ ID NO: 47 - DNA, 20 bp, Artificial Sequence
  Antisense Compound
agccgtcacagccggagcca

SEQ ID NO: 48 - DNA, 20 bp, Artificial Sequence
  Antisense Compound
gcagccgcagtgagaacggc

SEQ ID NO: 49 - DNA, 20 bp, Artificial Sequence
  Antisense Compound
tcaaagagccaggagaggca

SEQ ID NO: 50 - DNA, 20 bp, Artificial Sequence
  Antisense Compound
gagaaccctagtccagacca

SEQ ID NO: 51 - DNA, 20 bp, Artificial Sequence
  Antisense Compound
gatgaatactacaggctgcg

SEQ ID NO: 52 - DNA, 20 bp, Artificial Sequence
  Antisense Compound
gggccaccacgatgaatact

SEQ ID NO: 53 - DNA, 20 bp, Artificial Sequence
  Antisense Compound
gagacctctgcttgtctcct

SEQ ID NO: 54 - DNA, 20 bp, Artificial Sequence
  Antisense Compound
tgagaaatccaatagagacc

SEQ ID NO: 55 - DNA, 20 bp, Artificial Sequence
  Antisense Compound
ttcttcaggtgttgagaaat

SEQ ID NO: 56 - DNA, 20 bp, Artificial Sequence
  Antisense Compound
ggtttatttgaaatacaaca

SEQ ID NO: 57 - DNA, 20 bp, Artificial Sequence
  Antisense Compound
gcttcagtgtgaattctgcc

SEQ ID NO: 58 - DNA, 20 bp, Artificial Sequence
  Antisense Compound
agcagctctttctctgttta

SEQ ID NO: 59 - DNA, 20 bp, Artificial Sequence
  Antisense Compound
atatatgttcagtaccagtc

SEQ ID NO: 60 - DNA, 20 bp, Artificial Sequence
  Antisense Compound
agttcaaggtcagttgcaac

SEQ ID NO: 61 - DNA, 20 bp, Artificial Sequence
  Antisense Compound
gagagtgattcagttcaagg

SEQ ID NO: 62 - DNA, 20 bp, Artificial Sequence
  Antisense Compound
cccgagttcaagaacgtggg

SEQ ID NO: 63 - DNA, 20 bp, Artificial Sequence
  Antisense Compound
ctgtgaggatgtgctgcccg

SEQ ID NO: 64 - DNA, 20 bp, Artificial Sequence
  Antisense Compound
acagttcctagatttctgtg

SEQ ID NO: 65 - DNA, 20 bp, Artificial Sequence
  Antisense Compound
tgcaggagggatgactatga

SEQ ID NO: 66 - DNA, 20 bp, Artificial Sequence
  Antisense Compound
aaagcatgtttacagtgttt

SEQ ID NO: 67 - DNA, 20 bp, Artificial Sequence
  Antisense Compound
ccttcttattaaaagcatgt

SEQ ID NO: 68 - DNA, 20 bp, Artificial Sequence
  Antisense Compound
atgttcacatgatttcccaa

SEQ ID NO: 69 - DNA, 20 bp, Artificial Sequence
  Antisense Compound
gagcttgcagatttggagat

SEQ ID NO: 70 - DNA, 20 bp, Artificial Sequence
  Antisense Compound
tctacagtaaaacaggagct

SEQ ID NO: 71 - DNA, 20 bp, Artificial Sequence
  Antisense Compound
aaagattacatgaggagtgg

SEQ ID NO: 72 - DNA, 20 bp, Artificial Sequence
  Antisense Compound
actctcacagaaagaggagg

SEQ ID NO: 73 - DNA, 20 bp, Artificial Sequence
  Antisense Compound
ccactgagggactcaggact

SEQ ID NO: 74 - DNA, 20 bp, Artificial Sequence
  Antisense Compound
gggccgccctgaagaaggag

SEQ ID NO: 75 - DNA, 20 bp, Artificial Sequence
  Antisense Compound
gatggctgcgcccgaaaagg

SEQ ID NO: 76 - DNA, 20 bp, Artificial Sequence
  Antisense Compound
ccggcaagatggctgcgccc

SEQ ID NO: 77 - DNA, 20 bp, Artificial Sequence
  Antisense Compound
gggctgaatctgcggttaca

SEQ ID NO: 78 - DNA, 20 bp, Artificial Sequence
  Antisense Compound
tttgcacgcttcgttcatct

SEQ ID NO: 79 - DNA, 20 bp, Artificial Sequence
  Antisense Compound
gttcctcagtctgagcactt

SEQ ID NO: 80 - DNA, 20 bp, Artificial Sequence
  Antisense Compound
tcagtccctgttcctcagtc

SEQ ID NO: 81 - DNA, 20 bp, Artificial Sequence
  Antisense Compound
cacttcttaactcagtccct

SEQ ID NO: 82 - DNA, 20 bp, Artificial Sequence
  Antisense Compound
gatctgactggatcacgcca

SEQ ID NO: 83 - DNA, 20 bp, Artificial Sequence
  Antisense Compound
tcaggaggcttgatctgact

SEQ ID NO: 84 - DNA, 20 bp, Artificial Sequence
  Antisense Compound
ttctggaagactgctcctca

SEQ ID NO: 85 - DNA, 20 bp, Artificial Sequence
  Antisense Compound
gcgttccactgcagccttca

SEQ ID NO: 86 - DNA, 20 bp, Artificial Sequence
  Antisense Compound
tctggattgttcctgactta

SEQ ID NO: 87 - DNA, 20 bp, Artificial Sequence
  Antisense Compound
accttggacactgctaagct

SEQ ID NO: 88 - DNA, 20 bp, Artificial Sequence
  Antisense Compound
accttggacaccaggaggcc

SEQ ID NO: 89 - DNA, 20 bp, Artificial Sequence
  Antisense Compound
gctgttcctcctctgaatgg

SEQ ID NO: 90 - DNA, 20 bp, Artificial Sequence
  Antisense Compound
ctgcttgaagctgctcctca

SEQ ID NO: 91 - DNA, 20 bp, Artificial Sequence
  Antisense Compound
tgttcacatgatttctgtga

SEQ ID NO: 92 - DNA, 20 bp, Artificial Sequence
  Antisense Compound
tgttcctcctctgaatggat

SEQ ID NO: 93 - DNA, 20 bp, Artificial Sequence
  Antisense Compound
tcctctctacctgctaagct

SEQ ID NO: 94 - DNA, 20 bp, Artificial Sequence
  Antisense Compound
tgcccctgaccttggacaag

SEQ ID NO: 95 - DNA, 20 bp, Artificial Sequence
  Antisense Compound
gcctggattgttcctgactt

SEQ ID NO: 96 - DNA, 20 bp, Artificial Sequence
  Antisense Compound
agatttctctggaagctcac

SEQ ID NO: 97 - DNA, 20 bp, Artificial Sequence
  Antisense Compound
ctcctggtagatctcctgca

SEQ ID NO: 98 - DNA, 20 bp, Artificial Sequence
  Antisense Compound
ggaagctcacccactgcagc

SEQ ID NO: 99 - DNA, 20 bp, Artificial Sequence
  Antisense Compound
ccaggtgtacctgctcctca

SEQ ID NO: 100 - DNA, 20 bp, Artificial Sequence
  Antisense Compound
tgagtgacatcacaacgaca

SEQ ID NO: 101 - DNA, 20 bp, Artificial Sequence
  Antisense Compound
ttctggaagactgcagctgt

SEQ ID NO: 102 - DNA, 20 bp, Artificial Sequence
  Antisense Compound
agctgctgcagtcttggttc

SEQ ID NO: 103 - DNA, 20 bp, Artificial Sequence
  Antisense Compound
caagacaccctgctaagctc

SEQ ID NO: 104 - DNA, 20 bp, Artificial Sequence
  Antisense Compound
atggccaagacaccctgcta

SEQ ID NO: 105 - DNA, 20 bp, Artificial Sequence
  Antisense Compound
gaggagttgcagcaccaggg

SEQ ID NO: 106 - DNA, 20 bp, Artificial Sequence
  Antisense Compound
aaggagaggagttgcagcac

SEQ ID NO: 107 - DNA, 20 bp, Artificial Sequence
  Antisense Compound
caagagcgtgaaggagagga

SEQ ID NO: 108 - DNA, 20 bp, Artificial Sequence
  Antisense Compound
ccagccaagagcgtgaagga

SEQ ID NO: 109 - DNA, 20 bp, Artificial Sequence
  Antisense Compound
ggagcccagccaagagcgtg

SEQ ID NO: 110 - DNA, 20 bp, Artificial Sequence
  Antisense Compound
acacttggacaaggagccca

SEQ ID NO: 111 - DNA, 20 bp, Artificial Sequence
  Antisense Compound
ggatttctctgagagctcac

SEQ ID NO: 112 - DNA, 20 bp, Artificial Sequence
  Antisense Compound
ctgcagcttggatttctctg

SEQ ID NO: 113 - DNA, 20 bp, Artificial Sequence
  Antisense Compound
atctcctgcagcttggattt

SEQ ID NO: 114 - DNA, 20 bp, Artificial Sequence
  Antisense Compound
ggtagatctcctgcagcttg

SEQ ID NO: 115 - DNA, 20 bp, Artificial Sequence
  Antisense Compound
gtcagctcctggtagatctc

SEQ ID NO: 116 - DNA, 20 bp, Artificial Sequence
  Antisense Compound
agccttcagctgggtcagct

SEQ ID NO: 117 - DNA, 20 bp, Artificial Sequence
  Antisense Compound
actgcagccttcagctgggt

SEQ ID NO: 118 - DNA, 20 bp, Artificial Sequence
  Antisense Compound
tcacccactgcagccttcag

SEQ ID NO: 119 - DNA, 20 bp, Artificial Sequence
  Antisense Compound
gaagctcacccactgcagcc

SEQ ID NO: 120 - DNA, 20 bp, Artificial Sequence
  Antisense Compound
ctctggaagctcacccactg

SEQ ID NO: 121 - DNA, 20 bp, Artificial Sequence
  Antisense Compound
gatttctctggaagctcacc

SEQ ID NO: 122 - DNA, 20 bp, Artificial Sequence
  Antisense Compound
tggtagatctcctgcagctt

SEQ ID NO: 123 - DNA, 20 bp, Artificial Sequence
  Antisense Compound
gctcctggtagatctcctgc

SEQ ID NO: 124 - DNA, 20 bp, Artificial Sequence
  Antisense Compound
ggtcagctcctggtagatct

SEQ ID NO: 125 - DNA, 20 bp, Artificial Sequence
  Antisense Compound
agccgggtcagctcctggta

SEQ ID NO: 126 - DNA, 20 bp, Artificial Sequence
  Antisense Compound
ccttcagccgggtcagctcc

SEQ ID NO: 127 - DNA, 20 bp, Artificial Sequence
  Antisense Compound
tgcagccttcagccgggtca

SEQ ID NO: 128 - DNA, 20 bp, Artificial Sequence
  Antisense Compound
agccaggtcagctcctggta

SEQ ID NO: 129 - DNA, 20 bp, Artificial Sequence
  Antisense Compound
ccttcagccaggtcagctcc

SEQ ID NO: 130 - DNA, 20 bp, Artificial Sequence
  Antisense Compound
tgcagccttcagccaggtca

SEQ ID NO: 131 - DNA, 20 bp, Artificial Sequence
  Antisense Compound
ctgcatcttagatttctctg

SEQ ID NO: 132 - DNA, 20 bp, Artificial Sequence
  Antisense Compound
atctcctgcatcttagattt

SEQ ID NO: 133 - DNA, 20 bp, Artificial Sequence
  Antisense Compound
ggtagatctcctgcatctta

SEQ ID NO: 134 - DNA, 20 bp, Artificial Sequence
  Antisense Compound
tggtagatctcctgctgctt

SEQ ID NO: 135 - DNA, 20 bp, Artificial Sequence
  Antisense Compound
gttccactgcagccttcagc

SEQ ID NO: 136 - DNA, 20 bp, Artificial Sequence
  Antisense Compound
tttccttggaagaatgtcca

SEQ ID NO: 137 - DNA, 20 bp, Artificial Sequence
  Antisense Compound
aacagtttccttggaagaat

SEQ ID NO: 138 - DNA, 20 bp, Artificial Sequence
  Antisense Compound
cgctgggagttagacatgaa

SEQ ID NO: 139 - DNA, 20 bp, Artificial Sequence
  Antisense Compound
agttccgctgggagttagac

SEQ ID NO: 140 - DNA, 20 bp, Artificial Sequence
  Antisense Compound
gtgccagttccgctgggagt

SEQ ID NO: 141 - DNA, 20 bp, Artificial Sequence
  Antisense Compound
gagtcgtgccagttccgctg

SEQ ID NO: 142 - DNA, 20 bp, Artificial Sequence
  Antisense Compound
tgatggagtcgtgccagttc

SEQ ID NO: 143 - DNA, 20 bp, Artificial Sequence
  Antisense Compound
acgacgagctgggcccccac

SEQ ID NO: 144 - DNA, 20 bp, Artificial Sequence
  Antisense Compound
tgattacgacgagctgggcc

SEQ ID NO: 145 - DNA, 20 bp, Artificial Sequence
  Antisense Compound
acttttgattacgacgagct

SEQ ID NO: 146 - DNA, 20 bp, Artificial Sequence
  Antisense Compound
tcagcacttttgattacgac

SEQ ID NO: 147 - DNA, 20 bp, Artificial Sequence
  Antisense Compound
gctcctcagcacttttgatt

SEQ ID NO: 148 - DNA, 20 bp, Artificial Sequence
  Antisense Compound
gttctgctcctcagcacttt

SEQ ID NO: 149 - DNA, 20 bp, Artificial Sequence
  Antisense Compound
aggaagttctgctcctcagc

SEQ ID NO: 150 - DNA, 20 bp, Artificial Sequence
  Antisense Compound
cttctggaagactgcagctg

SEQ ID NO: 151 - DNA, 20 bp, Artificial Sequence
  Antisense Compound
ggttacttctggaagactgc

SEQ ID NO: 152 - DNA, 20 bp, Artificial Sequence
  Antisense Compound
gaagcggttacttctggaag

SEQ ID NO: 153 - DNA, 20 bp, Artificial Sequence
  Antisense Compound
caggtgaagcggttacttct

SEQ ID NO: 154 - DNA, 20 bp, Artificial Sequence
  Antisense Compound
ccatccaggtgaagcggtta

SEQ ID NO: 155 - DNA, 20 bp, Artificial Sequence
  Antisense Compound
aagtcccatccaggtgaagc

SEQ ID NO: 156 - DNA, 20 bp, Artificial Sequence
  Antisense Compound
tctgaaagtcccatccaggt

SEQ ID NO: 157 - DNA, 20 bp, Artificial Sequence
  Antisense Compound
ctgggcaacagaggtgagcc

SEQ ID NO: 158 - DNA, 20 bp, Artificial Sequence
  Antisense Compound
tgaagctgggcaacagaggt

SEQ ID NO: 159 - DNA, 20 bp, Artificial Sequence
  Antisense Compound
ctgcttgaagctgggcaaca

SEQ ID NO: 160 - DNA, 20 bp, Artificial Sequence
  Antisense Compound
caatactgcttgaagctggg

SEQ ID NO: 161 - DNA, 20 bp, Artificial Sequence
  Antisense Compound
tgttccaatactgcttgaag

SEQ ID NO: 162 - DNA, 20 bp, Artificial Sequence
  Antisense Compound
ccattgccactaaattccgc

SEQ ID NO: 163 - DNA, 20 bp, Artificial Sequence
  Antisense Compound
tccagccattgccactaaat

SEQ ID NO: 164 - DNA, 20 bp, Artificial Sequence
  Antisense Compound
tttttgcagatccagaattt

SEQ ID NO: 165 - DNA, 20 bp, Artificial Sequence
  Antisense Compound
gagattttgtgaaggtcgaa

SEQ ID NO: 166 - DNA, 20 bp, Artificial Sequence
  Antisense Compound
gaaggacagaatgggaccca

SEQ ID NO: 167 - DNA, 20 bp, Artificial Sequence
  Antisense Compound
gggccagaaaaacaagctct

SEQ ID NO: 168 - DNA, 20 bp, Artificial Sequence
  Antisense Compound
tatcctaatctccaaaagga

SEQ ID NO: 169 - DNA, 20 bp, Artificial Sequence
  Antisense Compound
agagtcccaaatcccagtaa

SEQ ID NO: 170 - DNA, 20 bp, Artificial Sequence
  Antisense Compound
gggaaggagaaccctagtcc

SEQ ID NO: 171 - DNA, 20 bp, Artificial Sequence
  Antisense Compound
caggacgacagcttcagtgt

SEQ ID NO: 172 - DNA, 20 bp, Artificial Sequence
  Antisense Compound
gaaacctaccaacagttcct

SEQ ID NO: 173 - DNA, 20 bp, Artificial Sequence
  Antisense Compound
cctccttgaatcttacttag

SEQ ID NO: 174 - DNA, 20 bp, Artificial Sequence
  Antisense Compound
tctgacagagactacagctt

SEQ ID NO: 175 - DNA, 20 bp, Artificial Sequence
  Antisense Compound
ttctcagtgccagaatctga

SEQ ID NO: 176 - DNA, 20 bp, Artificial Sequence
  Antisense Compound
tgtttcacagccacttctca

SEQ ID NO: 177 - DNA, 20 bp, Artificial Sequence
  Antisense Compound
tcttccccatttccttagaa

SEQ ID NO: 178 - DNA, 20 bp, Artificial Sequence
  Antisense Compound
tgtcagcagttcctcatcca

SEQ ID NO: 179 - DNA, 20 bp, Artificial Sequence
  Antisense Compound
tgatggagtgcctggtgtgg

SEQ ID NO: 180 - DNA, 20 bp, Artificial Sequence
  Antisense Compound
gaatttgtttggaagccaaa

SEQ ID NO: 181 - DNA, 20 bp, Artificial Sequence
  Antisense Compound
ccaggcaccctgtgaagcta

SEQ ID NO: 182 - DNA, 20 bp, Artificial Sequence
  Antisense Compound
aggaagagcacctgcagtgc

SEQ ID NO: 183 - DNA, 20 bp, Artificial Sequence
  Antisense Compound
gacaaggatgacaaccagca

SEQ ID NO: 184 - DNA, 20 bp, Artificial Sequence
  Antisense Compound
gttcttggtagacattcatc

SEQ ID NO: 185 - DNA, 20 bp, Artificial Sequence
  Antisense Compound
ccagccttcaactgggtcag

SEQ ID NO: 186 - DNA, 20 bp, Artificial Sequence
  Antisense Compound
aggagcggcacagtcgatct

SEQ ID NO: 187 - DNA, 20 bp, Artificial Sequence
  Antisense Compound
agtaacagcttccttggaag

SEQ ID NO: 188 - DNA, 20 bp, Artificial Sequence
  Antisense Compound
tggcagaatcattccaggac

SEQ ID NO: 189 - DNA, 20 bp, Artificial Sequence
  Antisense Compound
tcttgatgaccacaagttga

SEQ ID NO: 190 - DNA, 20 bp, Artificial Sequence
  Antisense Compound
tgtagcctctcttcttagaa

SEQ ID NO: 191 - DNA, 20 bp, Artificial Sequence
  Antisense Compound
catgtagactccttgctcat

SEQ ID NO: 192 - DNA, 20 bp, Artificial Sequence
  Antisense Compound
caggttgttaggttctcctt

SEQ ID NO: 193 - DNA, 20 bp, Artificial Sequence
  Antisense Compound
ccgtcatctctgaactctgc

SEQ ID NO: 194 - DNA, 20 bp, Artificial Sequence
  Antisense Compound
ctttttgcagatccagaatt

SEQ ID NO: 195 - DNA, 20 bp, Artificial Sequence
  Antisense Compound
gagttggccatcacttgcta

SEQ ID NO: 196 - DNA, 20 bp, Artificial Sequence
  Antisense Compound
tggtggagggagttggccat

SEQ ID NO: 197 - DNA, 20 bp, Artificial Sequence
  Antisense Compound
actggcatagctatgggtaa

SEQ ID NO: 198 - DNA, 20 bp, Artificial Sequence
  Antisense Compound
cttgtcaaggttatcaatgg

SEQ ID NO: 199 - DNA, 20 bp, Artificial Sequence
  Antisense Compound
tgcaggagacaagctacctg

SEQ ID NO: 200 - DNA, 20 bp, Artificial Sequence
  Antisense Compound
cattaatggtccaggagaca

SEQ ID NO: 201 - DNA, 20 bp, Artificial Sequence
  Antisense Compound
cactaacccaaagagaacct

SEQ ID NO: 202 - DNA, 20 bp, Artificial Sequence
  Antisense Compound
atgtctgtttacttcattaa

SEQ ID NO: 203 - DNA, 20 bp, Artificial Sequence
  Antisense Compound
aggaagacagaggagatgaa

SEQ ID NO: 204 - DNA, 20 bp, Artificial Sequence
  Antisense Compound
tcaattgcacatggcaatat

SEQ ID NO: 205 - DNA, 20 bp, Artificial Sequence
  Antisense Compound
aggtgtgcagggtagatgaa

SEQ ID NO: 206 - DNA, 20 bp, Artificial Sequence
  Antisense Compound
tcctgaaatcagagaagggt

SEQ ID NO: 207 - DNA, 20 bp, Artificial Sequence
  Antisense Compound
ttagtctataggttgagctc

SEQ ID NO: 208 - DNA, 20 bp, Artificial Sequence
  Antisense Compound
gactaatagttaaggaattg

SEQ ID NO: 209 - DNA, 20 bp, Artificial Sequence
  Antisense Compound
ggagtcagagaattaacaca

SEQ ID NO: 210 - DNA, 20 bp, Artificial Sequence
  Antisense Compound
ggctcaccatgtccacccag

SEQ ID NO: 211 - DNA, 20 bp, Artificial Sequence
  Antisense Compound
ttaatgagtaagttcaataa

SEQ ID NO: 212 - DNA, 20 bp, Artificial Sequence
  Antisense Compound
acttatatttatacatacat

SEQ ID NO: 213 - DNA, 20 bp, H. sapiens
tggggtgacatgagtgactc

SEQ ID NO: 214 - DNA, 20 bp, H. sapiens
ttggattccgacagactcga

SEQ ID NO: 215 - DNA, 20 bp, H. sapiens
tacaagagcttagcagggtg

SEQ ID NO: 216 - DNA, 20 bp, H. sapiens
ttggctgggctccttgtcca

SEQ ID NO: 217 - DNA, 20 bp, H. sapiens
gcttccagagaaatctaaga

SEQ ID NO: 218 - DNA, 20 bp, H. sapiens
aatcttgccaaattctggat

SEQ ID NO: 219 - DNA, 20 bp, H. sapiens
ttttaagctacagttccttc

SEQ ID NO: 220 - DNA, 20 bp, H. sapiens
tcctttagaaggctgggtcc

SEQ ID NO: 221 - DNA, 20 bp, H. sapiens
ggtggacttgtgttccaaga

SEQ ID NO: 222 - DNA, 20 bp, H. sapiens
attaggatatttgggttgcc

SEQ ID NO: 223 - DNA, 20 bp, H. sapiens
ccctttgatcggggacttac

SEQ ID NO: 224 - DNA, 20 bp, H. sapiens
acgactttgggatctagagg

SEQ ID NO: 225 - DNA, 20 bp, H. sapiens
ggatctagaggagctttggc

SEQ ID NO: 226 - DNA, 20 bp, H. sapiens
tggctccggctgtgacggct

SEQ ID NO: 227 - DNA, 20 bp, H. sapiens
gccgttctcactgcggctgc

SEQ ID NO: 228 - DNA, 20 bp, H. sapiens
tgcctctcctggctctttga

SEQ ID NO: 229 - DNA, 20 bp, H. sapiens
tggtctggactagggttctc

SEQ ID NO: 230 - DNA, 20 bp, H. sapiens
cgcagcctgtagtattcatc

SEQ ID NO: 231 - DNA, 20 bp, H. sapiens
agtattcatcgtggtggccc

SEQ ID NO: 232 - DNA, 20 bp, H. sapiens
aggagacaagcagaggtctc

SEQ ID NO: 233 - DNA, 20 bp, H. sapiens
ggtctctattggatttctca

SEQ ID NO: 234 - DNA, 20 bp, H. sapiens
atttctcaacacctgaagaa

SEQ ID NO: 235 - DNA, 20 bp, H. sapiens
tgttgtatttcaaataaacc

SEQ ID NO: 236 - DNA, 20 bp, H. sapiens
ggcagaattcacactgaagc

SEQ ID NO: 237 - DNA, 20 bp, H. sapiens
taaacagagaaagagctgct

SEQ ID NO: 238 - DNA, 20 bp, H. sapiens
gactggtactgaacatatat

SEQ ID NO: 239 - DNA, 20 bp, H. sapiens
gttgcaactgaccttgaact

SEQ ID NO: 240 - DNA, 20 bp, H. sapiens
ccttgaactgaatcactctc

SEQ ID NO: 241 - DNA, 20 bp, H. sapiens
cccacgttcttgaactcggg

SEQ ID NO: 242 - DNA, 20 bp, H. sapiens
cgggcagcacatcctcacag

SEQ ID NO: 243 - DNA, 20 bp, H. sapiens
cacagaaatctaggaactgt

SEQ ID NO: 244 - DNA, 20 bp, H. sapiens
tcatagtcatccctcctgca

SEQ ID NO: 245 - DNA, 20 bp, H. sapiens
aaacactgtaaacatgcttt

SEQ ID NO: 246 - DNA, 20 bp, H. sapiens
acatgcttttaataagaagg

SEQ ID NO: 247 - DNA, 20 bp, H. sapiens
ttgggaaatcatgtgaacat

SEQ ID NO: 248 - DNA, 20 bp, H. sapiens
atctccaaatctgcaagctc

SEQ ID NO: 249 - DNA, 20 bp, H. sapiens
agctcctgttttactgtaga

SEQ ID NO: 250 - DNA, 20 bp, H. sapiens
cctcctctttctgtgagagt

SEQ ID NO: 251 - DNA, 20 bp, H. sapiens
agtcctgagtccctcagtgg

SEQ ID NO: 252 - DNA, 20 bp, H. sapiens
ctccttcttcagggcggccc

SEQ ID NO: 253 - DNA, 20 bp, H. sapiens
ccttttcgggcgcagccatc

SEQ ID NO: 254 - DNA, 20 bp, H. sapiens
gggcgcagccatcttgccgg

SEQ ID NO: 255 - DNA, 20 bp, H. sapiens
tgtaaccgcagattcagccc

SEQ ID NO: 256 - DNA, 20 bp, H. sapiens
agatgaacgaagcgtgcaaa

SEQ ID NO: 257 - DNA, 20 bp, H. sapiens
aagtgctcagactgaggaac

SEQ ID NO: 258 - DNA, 20 bp, H. sapiens
gactgaggaacagggactga

SEQ ID NO: 259 - DNA, 20 bp, H. sapiens
agggactgagttaagaagtg

SEQ ID NO: 260 - DNA, 20 bp, H. sapiens
tggcgtgatccagtcagatc

SEQ ID NO: 261 - DNA, 20 bp, H. sapiens
agtcagatcaagcctcctga

SEQ ID NO: 262 - DNA, 20 bp, H. sapiens
tgaaggctgcagtggaacgc

SEQ ID NO: 263 - DNA, 20 bp, H. sapiens
taagtcaggaacaatccaga

SEQ ID NO: 264 - DNA, 20 bp, H. sapiens
tgaggagcagcttcaagcag

SEQ ID NO: 265 - DNA, 20 bp, H. sapiens
agcttagcaggtagagagga

SEQ ID NO: 266 - DNA, 20 bp, H. sapiens
aagtcaggaacaatccaggc

SEQ ID NO: 267 - DNA, 20 bp, H. sapiens
gtgagcttccagagaaatct

SEQ ID NO: 268 - DNA, 20 bp, H. sapiens
tgcaggagatctaccaggag

SEQ ID NO: 269 - DNA, 20 bp, H. sapiens
gctgcagtgggtgagcttcc

SEQ ID NO: 270 - DNA, 20 bp, H. sapiens
tgaggagcaggtacacctgg

SEQ ID NO: 271 - DNA, 20 bp, H. sapiens
acagctgcagtcttccagaa

SEQ ID NO: 272 - DNA, 20 bp, H. sapiens
ccctggtgctgcaactcctc

SEQ ID NO: 273 - DNA, 20 bp, H. sapiens
gtgctgcaactcctctcctt

SEQ ID NO: 274 - DNA, 20 bp, H. sapiens
tgggctccttgtccaagtgt

SEQ ID NO: 275 - DNA, 20 bp, H. sapiens
gtgagctctcagagaaatcc

SEQ ID NO: 276 - DNA, 20 bp, H. sapiens
cagagaaatccaagctgcag

SEQ ID NO: 277 - DNA, 20 bp, H. sapiens
aaatccaagctgcaggagat

SEQ ID NO: 278 - DNA, 20 bp, H. sapiens
caagctgcaggagatctacc

SEQ ID NO: 279 - DNA, 20 bp, H. sapiens
gagatctaccaggagctgac

SEQ ID NO: 280 - DNA, 20 bp, H. sapiens
agctgacccagctgaaggct

SEQ ID NO: 281 - DNA, 20 bp, H. sapiens
acccagctgaaggctgcagt

SEQ ID NO: 282 - DNA, 20 bp, H. sapiens
ctgaaggctgcagtgggtga

SEQ ID NO: 283 - DNA, 20 bp, H. sapiens
ggctgcagtgggtgagcttc

SEQ ID NO: 284 - DNA, 20 bp, H. sapiens
cagtgggtgagcttccagag

SEQ ID NO: 285 - DNA, 20 bp, H. sapiens
ggtgagcttccagagaaatc

SEQ ID NO: 286 - DNA, 20 bp, H. sapiens
aagctgcaggagatctacca

SEQ ID NO: 287 - DNA, 20 bp, H. sapiens
gcaggagatctaccaggagc

SEQ ID NO: 288 - DNA, 20 bp, H. sapiens
agatctaccaggagctgacc

SEQ ID NO: 289 - DNA, 20 bp, H. sapiens
taccaggagctgacccggct

SEQ ID NO: 290 - DNA, 20 bp, H. sapiens
ggagctgacccggctgaagg

SEQ ID NO: 291 - DNA, 20 bp, H. sapiens
tgacccggctgaaggctgca

SEQ ID NO: 292 - DNA, 20 bp, H. sapiens
taccaggagctgacctggct

SEQ ID NO: 293 - DNA, 20 bp, H. sapiens
ggagctgacctggctgaagg

SEQ ID NO: 294 - DNA, 20 bp, H. sapiens
tgacctggctgaaggctgca

SEQ ID NO: 295 - DNA, 20 bp, H. sapiens
cagagaaatctaagatgcag

SEQ ID NO: 296 - DNA, 20 bp, H. sapiens
aaatctaagatgcaggagat

SEQ ID NO: 297 - DNA, 20 bp, H. sapiens
taagatgcaggagatctacc

SEQ ID NO: 298 - DNA, 20 bp, H. sapiens
aagcagcaggagatctacca

SEQ ID NO: 299 - DNA, 20 bp, H. sapiens
gctgaaggctgcagtggaac

SEQ ID NO: 300 - DNA, 20 bp, H. sapiens
tggacattcttccaaggaaa

SEQ ID NO: 301 - DNA, 20 bp, H. sapiens
ttcatgtctaactcccagcg

SEQ ID NO: 302 - DNA, 20 bp, H. sapiens
actcccagcggaactggcac

SEQ ID NO: 303 - DNA, 20 bp, H. sapiens
cagcggaactggcacgactc

SEQ ID NO: 304 - DNA, 20 bp, H. sapiens
gaactggcacgactccatca

SEQ ID NO: 305 - DNA, 20 bp, H. sapiens
gtgggggcccagctcgtcgt

SEQ ID NO: 306 - DNA, 20 bp, H. sapiens
ggcccagctcgtcgtaatca

SEQ ID NO: 307 - DNA, 20 bp, H. sapiens
gtcgtaatcaaaagtgctga

SEQ ID NO: 308 - DNA, 20 bp, H. sapiens
aatcaaaagtgctgaggagc

SEQ ID NO: 309 - DNA, 20 bp, H. sapiens
aaagtgctgaggagcagaac

SEQ ID NO: 310 - DNA, 20 bp, H. sapiens
gctgaggagcagaacttcct

SEQ ID NO: 311 - DNA, 20 bp, H. sapiens
cagctgcagtcttccagaag

SEQ ID NO: 312 - DNA, 20 bp, H. sapiens
gcagtcttccagaagtaacc

SEQ ID NO: 313 - DNA, 20 bp, H. sapiens
cttccagaagtaaccgcttc

SEQ ID NO: 314 - DNA, 20 bp, H. sapiens
agaagtaaccgcttcacctg

SEQ ID NO: 315 - DNA, 20 bp, H. sapiens
taaccgcttcacctggatgg

SEQ ID NO: 316 - DNA, 20 bp, H. sapiens
gcttcacctggatgggactt

SEQ ID NO: 317 - DNA, 20 bp, H. sapiens
acctggatgggactttcaga

SEQ ID NO: 318 - DNA, 20 bp, H. sapiens
tgttgcccagcttcaagcag

SEQ ID NO: 319 - DNA, 20 bp, H. sapiens
cccagcttcaagcagtattg

SEQ ID NO: 320 - DNA, 20 bp, H. sapiens
cttcaagcagtattggaaca

SEQ ID NO: 321 - DNA, 20 bp, H. sapiens
gcggaatttagtggcaatgg

SEQ ID NO: 322 - DNA, 20 bp, H. sapiens
atttagtggcaatggctgga

SEQ ID NO: 323 - DNA, 20 bp, H. sapiens
ttcgaccttcacaaaatctc

SEQ ID NO: 324 - DNA, 20 bp, H. sapiens
tgggtcccattctgtccttc

SEQ ID NO: 325 - DNA, 20 bp, H. sapiens
agagcttgtttttctggccc

SEQ ID NO: 326 - DNA, 20 bp, H. sapiens
tccttttggagattaggata

SEQ ID NO: 327 - DNA, 20 bp, H. sapiens
ttactgggatttgggactct

SEQ ID NO: 328 - DNA, 20 bp, H. sapiens
ggactagggttctccttccc

SEQ ID NO: 329 - DNA, 20 bp, H. sapiens
acactgaagctgtcgtcctg

SEQ ID NO: 330 - DNA, 20 bp, H. sapiens
aggaactgttggtaggtttc

SEQ ID NO: 331 - DNA, 20 bp, M. musculus
ctaagtaagattcaaggagg

SEQ ID NO: 332 - DNA, 20 bp, M. musculus
tcagattctggcactgagaa

SEQ ID NO: 333 - DNA, 20 bp, M. musculus
tgagaagtggctgtgaaaca

SEQ ID NO: 334 - DNA, 20 bp, M. musculus
ttctaaggaaatggggaaga

SEQ ID NO: 335 - DNA, 20 bp, M. musculus
tggatgaggaactgctgaca

SEQ ID NO: 336 - DNA, 20 bp, M. musculus
ccacaccaggcactccatca

SEQ ID NO: 337 - DNA, 20 bp, M. musculus
tttggcttccaaacaaattc

SEQ ID NO: 338 - DNA, 20 bp, M. musculus
tagcttcacagggtgcctgg

SEQ ID NO: 339 - DNA, 20 bp, M. musculus
gcactgcaggtgctcttcct

SEQ ID NO: 340 - DNA, 20 bp, M. musculus
tgctggttgtcatccttgtc

SEQ ID NO: 341 - DNA, 20 bp, M. musculus
gatgaatgtctaccaagaac

SEQ ID NO: 342 - DNA, 20 bp, M. musculus
ctgacccagttgaaggctgg

SEQ ID NO: 343 - DNA, 20 bp, M. musculus
agatcgactgtgccgctcct

SEQ ID NO: 344 - DNA, 20 bp, M. musculus
cttccaaggaagctgttact

SEQ ID NO: 345 - DNA, 20 bp, M. musculus
gtcctggaatgattctgcca

SEQ ID NO: 346 - DNA, 20 bp, M. musculus
tcaacttgtggtcatcaaga

SEQ ID NO: 347 - DNA, 20 bp, M. musculus
ttctaagaagagaggctaca

SEQ ID NO: 348 - DNA, 20 bp, M. musculus
atgagcaaggagtctacatg

SEQ ID NO: 349 - DNA, 20 bp, M. musculus
aaggagaacctaacaacctg

SEQ ID NO: 350 - DNA, 20 bp, M. musculus
gcagagttcagagatgacgg

SEQ ID NO: 351 - DNA, 20 bp, M. musculus
aattctggatctgcaaaaag

SEQ ID NO: 352 - DNA, 20 bp, M. musculus
tagcaagtgatggccaactc

SEQ ID NO: 353 - DNA, 20 bp, M. musculus
atggccaactccctccacca

SEQ ID NO: 354 - DNA, 20 bp, M. musculus
ttacccatagctatgccagt

SEQ ID NO: 355 - DNA, 20 bp, M. musculus
ccattgataaccttgacaag

SEQ ID NO: 356 - DNA, 20 bp, M. musculus
caggtagcttgtctcctgca

SEQ ID NO: 357 - DNA, 20 bp, M. musculus
tgtctcctggaccattaatg

SEQ ID NO: 358 - DNA, 20 bp, M. musculus
aggttctctttgggttagtg

SEQ ID NO: 359 - DNA, 20 bp, M. musculus
ttaatgaagtaaacagacat

SEQ ID NO: 360 - DNA, 20 bp, M. musculus
ttcatctcctctgtcttcct

SEQ ID NO: 361 - DNA, 20 bp, M. musculus
atattgccatgtgcaattga

SEQ ID NO: 362 - DNA, 20 bp, M. musculus
ttcatctaccctgcacacct

SEQ ID NO: 363 - DNA, 20 bp, M. musculus
acccttctctgatttcagga

SEQ ID NO: 364 - DNA, 20 bp, M. musculus
gagctcaacctatagactaa

SEQ ID NO: 365 - DNA, 20 bp, M. musculus
caattccttaactattagtc

SEQ ID NO: 366 - DNA, 20 bp, M. musculus
tgtgttaattctctgactcc

SEQ ID NO: 367 - DNA, 20 bp, M. musculus
ctgggtggacatggtgagcc

SEQ ID NO: 368 - DNA, 20 bp, M. musculus
ttattgaacttactcattaa

SEQ ID NO: 369 - DNA, 20 bp, M. musculus
atgtatgtataaatataagt

SEQ ID NO: 370 - DNA, 20 bp, Artificial Sequence
  Antisense Compound
tcccgcctgtgacatgcatt

(19) United States
(12) Patent Application Publication
(10) Pub. No.: US 2005/0182004 A1
(43) Pub. Date: Aug. 18, 2005

(54) Modulation of DC-SIGN expression

(76) Inventors: C. Frank Bennett, Carlsbad, CA (US); Kenneth W. Dobie, Del Mar, CA (US); Susan Gregory, San Diego, CA (US)

Correspondence Address: FENWICK & WEST LLP, 801 CALIFORNIA STREET, MOUNTAIN VIEW, CA, 94014, US

(21) Appl. No.: 10/776,478

(22) Filed: Feb. 11, 2004

(51) Int. Cl. A61K 48/00; C07H 21/02; C07H 21/04

(52) U.S. Cl. 514/44; 536/23.1

(57) ABSTRACT

Compounds, compositions and methods are provided for modulating the expression of DC-SIGN. The compositions comprise oligonucleotides, targeted to nucleic acid encoding DC-SIGN. Methods of using these compounds for modulation of DC-SIGN expression and for diagnosis and treatment of diseases and conditions associated with expression of DC-SIGN are provided.

### FIELD

The present invention provides compositions and methods for modulating the expression of DC-SIGN. In particular, this invention relates to antisense compounds, particularly oligonucleotide compounds, which hybridize with nucleic acid molecules encoding DC-SIGN. Such compounds are shown herein to modulate the expression of DC-SIGN.

### BACKGROUND

Dendritic cells (DCs) are professional antigen-presenting cells that play a central part in the initiation of adaptive immune responses. DCs convert antigens from foreign cells and infectious microorganisms into short peptides that are presented on the plasma membrane bound to the major histocompatibility complex (MHC) receptors. After antigen uptake, the immature DCs acquire the capacity to migrate from the periphery to the T cell areas of secondary lymphoid organs where they attract resting T cells and present their antigens, thus causing T cell activation and subsequent immune responses.

Early studies showed that DCs and T cells adhere to each other in an antigen-independent manner. The initial contact between DCs and resting T cells has been shown to require interaction of cell surface proteins including the integrin receptor ICAM-3. ICAM-3 is expressed at high levels on resting T-cells, and antibodies to ICAM-3 trigger intracellular calcium elevation and induction of tyrosine phosphorylation, indicative of T-cell activation via these receptors (Geijtenbeek et al., Cell, 2000, 100, 575-585).

Using a flow cytometric assay, DCs were found to interact with ICAM-3 coated beads through an integrin-independent mechanism that requires calcium. The DC receptor for this interaction was therefore designated DC-SIGN (DC-specific ICAM-3 grabbing nonintegrin; also known as membrane-associated lectin type-C, CD209, CD209 antigen, CDSIGN, and DC-SIGN1) (Geijtenbeek et al., Cell, 2000, 100, 575-585).

DC-SIGN is a 44 kDa protein which is identical to the HIV-1 gpl20 binding C-type lectin. It is a type II transmembrane protein consisting of 404 amino acids. The N-terminal cytoplasmic domain is separated by a hydrophobic stretch of amino acids from a region that consists of several tandem repeats of nearly identical sequence. The extracellular C-terminal region is homologous to calcium dependent lectins, and the protein contains a motif that binds two calcium ions and a mannose. ICAM-3 binding depends on the binding of calcium and is at least partially dependent on mannose moieties (Geijtenbeek et al., Cell, 2000, 100, 575-585).

Antibodies to DC-SIGN or ICAM-3 inhibited DC-induced proliferation of resting T cells in vitro, signifying the importance of DC-SIGN/ICAM-3 interaction in the initiation of a primary immune response. DC-SIGN also binds the HIV-1 envelope glycoprotein gp120. The capture of HIV-1 through DC-SIGN by DCs in the initial sites of HIV infection facilitates the infection of T cells (Geijtenbeek et al., Cell, 2000, 100, 575-585).

Expression of DC-SIGN is consistent with its role in immunity. Both immature and mature monocyte-derived and a subpopulation of CD34⁺ bone marrow-derived DC express DC-SIGN. In situ, DC-SIGN is expressed by DC subsets present in the T cell area of tonsils, lymph nodes, and spleen. It is also expressed in mucosal tissues such as rectum, uterus, and cervix (Geijtenbeek et al., Cell, 2000, 100, 575-585).

The DC-SIGN/ICAM-3 interaction is thought to mediate the initial contact between DCs and resting T cells. Presumably, this interaction transiently stabilizes the cell-cell contact, allowing the T-cell receptor to contact the MHC-peptide complex on the DC surface. The activated T-cells subsequently respond to antigens presented by other cells (Geijtenbeek et al., Cell, 2000, 100, 575-585).

The role of DC-SIGN in primary immune responses makes it an attractive target for therapeutic and investigative strategies aimed at immune responsiveness. Consequently, there remains a need for agents capable of effectively modulating DC-SIGN function.

Antisense technology is an effective means for reducing the expression of specific gene products and may therefore prove to be uniquely useful in a number of therapeutic, diagnostic, and research applications for the modulation of DC-SIGN expression.

The present invention provides compositions and methods for modulating DC-SIGN expression.

### SUMMARY

The present invention is directed to antisense compounds, especially nucleic acid and nucleic acid-like oligomers, which are targeted to a nucleic acid encoding DC-SIGN, and which modulate the expression of DC-SIGN. Pharmaceutical and other compositions comprising the compounds are also provided. Further provided are methods of screening for modulators of DC-SIGN and methods of modulating the expression of DC-SIGN in cells, tissues or animals comprising contacting said cells, tissues or animals with one or more of the compounds or compositions. Methods of treating an animal, particularly a human, suspected of having or being prone to a disease or condition associated with expression of DC-SIGN are also set forth herein. Such methods comprise administering a therapeutically or prophylactically effective amount of one or more of the compounds or compositions to the person in need of treatment.

### BRIEF DESCRIPTION OF THE DRAWINGS

FIG. 1 is a graph showing the effect of two intratracheally (IT) administered antisense oligonucleotides targeted to nucleic acid encoding DC-SIGN, ISIS 290127 and ISIS 290135 in the mouse ovalbumin (OVA)-induced asthma model. Mch=methacholine; Penh=arbitrary units which measure airway hyperresponsiveness. Naïve mice were not sensitized with ovalbumin.

FIG. 2 is a graph showing the effect of IT administered DC-SIGN antisense oligonucleotides on the recruitment of eosinophils as measured in bronchaleveolar lavage (BAL) fluid in the mouse OVA-induced model of asthma.

FIG. 3 is a graph showing the effect of IT-administered DC-SIGN antisense oligonucleotides on pulmonary mucus production in the mouse OVA-induced asthma model. PAS=periodic acid Schiff reagent which stains mucus.

FIG. 4 is a graph showing the effect of two aerosol administered antisense oligonucleotides targeted to nucleic acid encoding DC-SIGN, ISIS 290129 and ISIS 290135 in the mouse ovalbumin (OVA)-induced asthma model.

FIG. 5 is a graph showing the effect of aerosol administered DC-SIGN antisense oligonucleotides on the recruitment of eosinophils as measured in bronchaleveolar lavage (BAL) fluid in the mouse OVA-induced model of asthma.

### DETAILED DESCRIPTION

A. Overview

The present invention employs antisense compounds, for example oligonucleotides and similar species, for use in modulating the function or effect of nucleic acid molecules encoding DC-SIGN. This is accomplished by providing oligonucleotides which specifically hybridize with one or more nucleic acid molecules encoding DC-SIGN.

Such modulation is desirable for treating various inflammatory or autoimmune disorders or diseases, or disorders or diseases with an inflammatory component such as asthma, juvenile diabetes mellitus, myasthenia gravis, Graves' disease, rheumatoid arthritis, allograft rejection, inflammatory bowel disease, multiple sclerosis, psoriasis, lupus erythematosus, systemic lupus erythematosus, diabetes, multiple sclerosis, contact dermatitis, rhinitis, various allergies, and cancers and their metastases. Such modulation is further desirable for preventing or modulating the development of such diseases or disorders in an animal suspected of being, or known to be, prone to such diseases or disorders.

As used herein, the terms “target nucleic acid” and “nucleic acid molecule encoding DC-SIGN” have been used for convenience to encompass DNA encoding DC-SIGN, RNA (including pre-mRNA and mRNA or portions thereof) transcribed from such DNA, and also cDNA derived from such RNA. The hybridization of a compound of this invention with its target nucleic acid is generally referred to as “antisense”. Consequently, one mechanism believed to be included in the practice of some embodiments described herein is referred to as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, specific nucleic acid molecules and their functions are targeted for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. One result of such interference with target nucleic acid function is modulation of the expression of DC-SIGN. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is one form of modulation of expression and mRNA is one example of a target nucleic acid.

In the context of this invention, “hybridization” means the pairing of complementary strands of oligomeric compounds. In the present invention, the predominant mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

In the present invention the phrase “stringent hybridization conditions” or “stringent conditions” refers to conditions under which a compound will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomeric compounds and the assays in which they are being investigated.

“Complementary,” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). In one embodiment, the antisense compounds of the present invention comprise at least 70%, or at least 75%, or at least 80%, or at least 85% sequence complementarity to a target region within the target nucleic acid, in another embodiment they comprise at least 90% sequence complementarity in yet another embodiment comprise at least 95% or at least 99% sequence complementarity to the target region within the target nucleic acid sequence to which they are targeted. For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, homology, sequence identity or complementarity, between the oligomeric and target is between about 50% to about 60%. In some embodiments, homology, sequence identity or complementarity, is between about 60% to about 70%. In some embodiments, homology, sequence identity or complementarity, is between about 70% and about 80%. In more embodiments, homology, sequence identity or complementarity, is between about 80% and about 90%. In some embodiments, homology, sequence identity or complementarity, is about 90%, about 92%, about 94%, about 95%, about 96%, about 97%, about 98%, about 99% or about 100%.

B. Compounds

As used herein, “antisense compounds” include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds may elicit the action of one or more enzymes or structural proteins to effect modification of the target nucleic acid.

One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the predominant form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

The first evidence that dsRNA could lead to gene silencing in animals came in 1995 from work in the nematode, Caenorhabditis elegans (Guo and Kempheus, Cell, 1995, 81, 611-620). Montgomery et al. have shown that the primary interference effects of dsRNA are posttranscriptional (Montgomery et al., Proc. Natl. Acad. Sci. USA, 1998, 95, 15502-15507). The posttranscriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA levels (Fire et al., Nature, 1998, 391, 806-811). Recently, it has been shown that it is, in fact, the single-stranded RNA oligomers of antisense polarity of the dsRNAs which are the potent inducers of RNAi (Tijsterman et al., Science, 2002, 295, 694-697).

The antisense compounds of the present invention also include modified compounds in which a different base is present at one or more of the nucleotide positions in the compound. For example, if the first nucleotide is an adenosine, modified compounds may be produced which contain thymidine, guanosine or cytidine at this position. This may be done at any of the positions of the antisense compound. These compounds are then tested using the methods described herein to determine their ability to inhibit expression of DC-SIGN mRNA.

In the context of this invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often used in place of native forms because of desirable properties such as, for example, enhanced cellular uptake, enhanced affinity for a target nucleic acid and increased stability in the presence of nucleases.

While oligonucleotides are one form of the antisense compounds of this invention, the present invention comprehends other families of antisense compounds as well, including but not limited to oligonucleotide analogs and mimetics such as those described herein.

The antisense compounds in accordance with this invention preferably comprise from about 8 to about 80 nucleobases (i.e. from about 8 to about 80 linked nucleosides). One of ordinary skill in the art will appreciate that the invention embodies compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

In one embodiment, the antisense compounds are 12 to 50 nucleobases in length. One having ordinary skill in the art will appreciate that this embodies compounds of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In another embodiment, the antisense compounds are 15 to 30 nucleobases in length. One having ordinary skill in the art will appreciate that this embodies compounds of 15, 16, 17, 18, 19, 20, 21, 22, 23, 24, 25, 26, 27, 28, 29, or 30 nucleobases in length.

Particularly compounds are oligonucleotides from about 12 to about 50 nucleobases, even more preferably those comprising from about 15 to about 30 nucleobases.

Antisense compounds 8-80 nucleobases in length comprising a stretch of at least eight (8) consecutive nucleobases selected from within the illustrative antisense compounds are considered to be suitable antisense compounds as well.

Exemplary antisense compounds include oligonucleotide sequences that comprise at least the 8 consecutive nucleobases from the 5′-terminus of one of the illustrative antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about 8 to about 80 nucleobases). Similarly antisense compounds are represented by oligonucleotide sequences that comprise at least the 8 consecutive nucleobases from the 3′-terminus of one of the illustrative antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide contains about 8 to about 80 nucleobases). It is also understood that antisense compounds may be represented by oligonucleotide sequences that comprise at least 8 consecutive nucleobases from an internal portion of the sequence of an illustrative antisense compound, and may extend in either or both directions until the oligonucleotide contains about 8 to about 80 nucleobases.

One having skill in the art armed with the antisense compounds illustrated herein will be able, without undue experimentation, to identify further antisense compounds.

C. Targets

“Targeting” an antisense compound to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent. In the present invention, the target nucleic acid encodes DC-SIGN.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. As used herein, “start codon” and “translation initiation codon” refer to the codon or codons that are used in vivo to initiate translation of an mRNA transcribed from a gene encoding DC-SIGN, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the antisense compounds of the present invention.

The open reading frame (ORF) or “coding region,” which is known in the art to refer to the region between the translation initiation codon and the translation termination codon, is also a region which may be targeted effectively. Within the context of the present invention, one region region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation-initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. This 5′ cap region may also be targeted.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also examples of suitable target sites. mRNA transcripts produced via the process of splicing of two (or more) mRNAs from different gene sources are known as “fusion transcripts”. It is also known that introns can be effectively targeted using antisense compounds targeted to, for example, DNA or pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. The types of variants described herein are also target nucleic acids.

The locations on the target nucleic acid to which the preferred antisense compounds hybridize are hereinbelow referred to as “preferred target segments.” As used herein the term “preferred target segment” is defined as at least an 8-nucleobase portion of a target region to which an active antisense compound is targeted. While not wishing to be bound by theory, it is presently believed that these target segments represent portions of the target nucleic acid which are accessible for hybridization.

While the specific sequences of certain preferred target segments are set forth herein, one of skill in the art will recognize that these serve to illustrate and describe particular embodiments within the scope of the present invention. Additional preferred target segments may be identified by one having ordinary skill.

Target segments 8-80 nucleobases in length comprising a stretch of at least eight (8) consecutive nucleobases selected from within the illustrative preferred target segments are considered to be suitable for targeting as well.

Target segments can include DNA or RNA sequences that comprise at least the 8 consecutive nucleobases from the 5′-terminus of one of the illustrative preferred target segments (the remaining nucleobases being a consecutive stretch of the same DNA or RNA beginning immediately upstream of the 5′-terminus of the target segment and continuing until the DNA or RNA contains about 8 to about 80 nucleobases). Similarly preferred target segments are represented by DNA or RNA sequences that comprise at least the 8 consecutive nucleobases from the 3′-terminus of one of the illustrative preferred target segments (the remaining nucleobases being a consecutive stretch of the same DNA or RNA beginning immediately downstream of the 3′-terminus of the target segment and continuing until the DNA or RNA contains about 8 to about 80 nucleobases). It is also understood that preferred antisense target segments may be represented by DNA or RNA sequences that comprise at least 8 consecutive nucleobases from an internal portion of the sequence of an illustrative preferred target segment, and may extend in either or both directions until the oligonucleotide contains about 8 to about 80 nucleobases. One having skill in the art armed with the preferred target segments illustrated herein will be able, without undue experimentation, to identify further preferred target segments.

Once one or more target regions, segments or sites have been identified, antisense compounds are chosen which are sufficiently complementary to the target, i.e., hybridize sufficiently well and with sufficient specificity, to give the desired effect.

The oligomeric antisense compounds may also be targeted to regions of the target nucleobase sequence (e.g., such as those disclosed in Example 13) comprising nucleobases 1-80, 81-160, 161-240, 241-320, 321-400, 401-480, 481-560, 561-640, 641-720, 721-800, 801-880, 881-960, 961-1040, 1041-1120, 1121-1200, 1201-1280, 1281-1360, 1361-1440, 1441-1520, 1521-1600, 1601-1680, 1681-1760, 1761-1840, 1841-1920, 1921-2000, 2001-2080, 2081-2160, 2161-2240, 2241-2320, 2321-2400, 2401-2480, 2481-2560, 2561-2640, 2641-2720, 2721-2800, 2801-2880, 2881-2960, 2961-3040, 3041-3120, 3121-3200, 3201-3280, 3281-3360, 3361-3440, 3441-3520, 3521-3600, 3601-3680, 3681-3760, 3761-3840, 3841-3920, 3921-4000, 4001-4080, 4081-4160, 4161-4240, 4241-4266, or any combination thereof.

D. Screening and Target Validation

In a further embodiment, the “preferred target segments” identified herein may be employed in a screen for additional compounds that modulate the expression of DC-SIGN. “Modulators” are those compounds that decrease or increase the expression of a nucleic acid molecule encoding DC-SIGN and which comprise at least an 8-nucleobase portion which is complementary to a preferred target segment. The screening method comprises the steps of contacting a preferred target segment of a nucleic acid molecule encoding DC-SIGN with one or more candidate modulators, and selecting for one or more candidate modulators which decrease or increase the expression of a nucleic acid molecule encoding DC-SIGN. Once it is shown that the candidate modulator or modulators are capable of modulating (e.g. either decreasing or increasing) the expression of a nucleic acid molecule encoding DC-SIGN, the modulator may then be employed in further investigative studies of the function of DC-SIGN, or for use as a research, diagnostic, or therapeutic agent in accordance with the present invention.

The preferred target segments of the present invention may be also be combined with their respective complementary antisense compounds of the present invention to form stabilized double-stranded (duplexed) oligonucleotides.

Such double stranded oligonucleotide moieties have been shown in the art to modulate target expression and regulate translation as well as RNA processsing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

The antisense compounds of the present invention can also be applied in the areas of drug discovery and target validation. The present invention comprehends the use of the compounds and preferred target segments identified herein in drug discovery efforts to elucidate relationships that exist between DC-SIGN and a disease state, phenotype, or condition. These methods include detecting or modulating DC-SIGN comprising contacting a sample, tissue, cell, or organism with the compounds of the present invention, measuring the nucleic acid or protein level of DC-SIGN and/or a related phenotypic or chemical endpoint at some time after treatment, and optionally comparing the measured value to a non-treated sample or sample treated with a further compound. These methods can also be performed in parallel or in combination with other experiments to determine the function of unknown genes for the process of target validation or to determine the validity of a particular gene product as a target for treatment or prevention of a particular disease, condition, or phenotype.

E. Kits, Research Reagents, Diagnostics, and Therapeutics

The antisense compounds of the present invention can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the compounds of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U. S. A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The antisense compounds described herein are useful for research and diagnostics, because these compounds hybridize to nucleic acids encoding DC-SIGN. For example, oligonucleotides that are shown to hybridize with such efficiency and under such conditions as disclosed herein as to be effective DC-SIGN inhibitors will also be effective primers or probes under conditions favoring gene amplification or detection, respectively. These primers and probes are useful in methods requiring the specific detection of nucleic acid molecules encoding DC-SIGN and in the amplification of said nucleic acid molecules for detection or for use in further studies of DC-SIGN. Hybridization of the antisense oligonucleotides, particularly the primers and probes, with a nucleic acid encoding DC-SIGN can be detected by means known in the art. Such means may include conjugation of an enzyme to the oligonucleotide, radiolabelling of the oligonucleotide or any other suitable detection means. Kits using such detection means for detecting the level of DC-SIGN in a sample may also be prepared.

The specificity and sensitivity of antisense is also harnessed by those of skill in the art for therapeutic uses. Antisense compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense compounds can be useful therapeutic modalities that can be configured to be useful in treatment regimes for the treatment of cells, tissues and animals, especially humans.

For therapeutics, an animal, preferably a human, suspected of having a disease or disorder which can be treated by modulating the expression of DC-SIGN is treated by administering antisense compounds in accordance with this invention. For example, in one non-limiting embodiment, the methods comprise the step of administering to the animal in need of treatment, a therapeutically effective amount of a DC-SIGN inhibitor. The DC-SIGN inhibitors of the present invention effectively inhibit the activity of the DC-SIGN protein or inhibit the expression of the DC-SIGN protein. In one embodiment, the activity or expression of DC-SIGN in an animal is inhibited by about 10%. Preferably, the activity or expression of DC-SIGN in an animal is inhibited by about 30%. More preferably, the activity or expression of DC-SIGN in an animal is inhibited by 50% or more. Thus, the oligomeric antisense compounds modulate expression of DC-SIGN mRNA by at least 10%, by at least 20%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100%.

For example, the reduction of the expression of DC-SIGN may be measured in serum, adipose tissue, liver or any other body fluid, tissue or organ of the animal. Preferably, the cells contained within said fluids, tissues or organs being analyzed contain a nucleic acid molecule encoding DC-SIGN protein and/or the DC-SIGN protein itself.

The antisense compounds can be utilized in pharmaceutical compositions by adding an effective amount of a compound to a suitable pharmaceutically acceptable diluent or carrier. Use of the compounds and methods of the invention may also be useful prophylactically.

F. Modifications

As is known in the art, a nucleoside is a base-sugar combination. The base portion of the nucleoside is normally a heterocyclic base sometimes referred to as a “nucleobase” or simply a “base”. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally more prevalent. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Modified Internucleoside Linkages (Backbones)

Specific examples of antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Modified oligonucleotide backbones containing a phosphorus atom therein include, for example, phosphorothioates, chiral phosphorothioates, phosphorodithioates, phosphotriesters, aminoalkylphosphotriaminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. No.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Modified Sugar and Internucleoside Linkages—Mimetics

In other antisense compounds, e.g., oligonucleotide mimetics, both the sugar and the internucleoside linkage (i.e. the backbone), of the nucleotide units are replaced with novel groups. The nucleobase units are maintained for hybridization with an appropriate target nucleic acid. One such compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science, 1991, 254, 1497-1500.

Other embodiments are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. In another embodiment, oligonucleotides have morpholino backbone structures of the above-referenced U.S. Pat. No. 5,034,506.

Modified Sugars

Modified antisense compounds may also contain one or more substituted sugar moieties. Antisense compounds, preferably antisense oligonucleotides, may comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Some emdiments are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. One modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-aminoethoxy-ethyl or 2′-DMAEOE), i.e., 2′-O—CH₂—O—CH₂—N(CH₃)₂, also described in examples hereinbelow.

Other modifications include 2′-methoxy (2′-O—CH₃), 2′aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Antisense compounds may also have sugar mimetics such as cyclobutyl moieties in place of the pentofuranosyl sugar. Representative United States patents that teach the preparation of such modified sugar structures include, but are not limited to, U.S. Pat. Nos.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

A further preferred modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is a methylene (—CH₂—)ₙ group bridging the 2′ oxygen atom and the 4′ carbon atom wherein n is 1 or 2. LNAs and preparation thereof are described in WO 98/39352 and WO 99/14226.

Natural and Modified Nucleobases

Antisense compounds may also include nucleobase (often referred to in the art as heterocyclic base or simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the compounds. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C., even more particularly when combined with 2′-O-methoxyethyl sugar modifications, and are another embodiment.

Representative United States patents that teach the preparation of certain of the above noted modified nucleobases as well as other modified nucleobases include, but are not limited to, the above noted U.S. Pat. No. 3,687,808, as well as U.S. Pat. Nos.: 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Conjugates

Another modification of the antisense compounds involves chemically linking to the antisense compound one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application PCT/US92/09196, filed Oct. 23, 1992, and U.S. Pat. No. 6,287,860, the entire disclosure of which are incorporated herein by reference. Conjugate moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Antisense compounds may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos.: 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

Chimeric Compounds

It is not necessary for all positions in a given compound to be uniformly modified, and in fact more than one of the aforementioned modifications may be incorporated in a single compound or even at a single nucleoside within an oligonucleotide.

The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. Chimeric antisense oligonucleotides are thus a form of antisense compound. These oligonucleotides typically contain at least one region wherein the oligonucleotide is modified so as to confer upon the oligonucleotide increased resistance to nuclease degradation, increased cellular uptake, increased stability and/or increased binding affinity for the target nucleic acid. An additional region of the oligonucleotide may serve as a substrate for enzymes capable of cleaving RNA:DNA or RNA:RNA hybrids. By way of example, RNAse H is a cellular endonuclease which cleaves the RNA strand of an RNA:DNA duplex. Activation of RNase H, therefore, results in cleavage of the RNA target, thereby greatly enhancing the efficiency of oligonucleotide-mediated inhibition of gene expression. The cleavage of RNA:RNA hybrids can, in like fashion, be accomplished through the actions of endoribonucleases, such as RNAseL which cleaves both cellular and viral RNA. Cleavage of the RNA target can be routinely detected by gel electrophoresis and, if necessary, associated nucleic acid hybridization techniques known in the art.

Chimeric antisense compounds may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos.: 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

G. Formulations

The compounds described herein may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos.: 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds encompass any pharmaceutically acceptable salts, esters, or salts of such esters, or any other compound which, upon administration to an animal, including a human, is capable of providing (directly or indirectly) the biologically active metabolite or residue thereof.

The term “pharmaceutically acceptable salts” refers to physiologically and pharmaceutically acceptable salts of the compounds: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety.

The present invention also includes pharmaceutical compositions and formulations which include the antisense compounds. The pharmaceutical compositions may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The pharmaceutical formulations, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions may be formulated into any of many possible dosage forms such as, but not limited to, tablets, capsules, gel capsules, liquid syrups, soft gels, suppositories, and enemas. The compositions may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions include, but are not limited to, solutions, emulsions, foams and liposome-containing formulations. The pharmaceutical compositions and formulations may comprise one or more penetration enhancers, carriers, excipients or other active or inactive ingredients.

Emulsions are typically heterogenous systems of one liquid dispersed in another in the form of droplets usually exceeding 0.1 μm in diameter. Emulsions may contain additional components in addition to the dispersed phases, and the active drug which may be present as a solution in either the aqueous phase, oily phase or itself as a separate phase. Microemulsions are included as an embodiment of the present invention. Emulsions and their uses are well known in the art and are further described in U.S. Pat. No. 6,287,860, which is incorporated herein in its entirety.

Formulations include liposomal formulations. As used herein, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions may also include surfactants. The use of surfactants in drug products, formulations and in emulsions is well known in the art. Surfactants and their uses are further described in U.S. Pat. No. 6,287,860, which is incorporated herein in its entirety.

In one embodiment, various penetration enhancers are employed to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the oligonucleotides are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, oligonucleotides may be encapsulated within liposomes or may form complexes thereto, in particular to cationic liposomes. Alternatively, oligonucleotides may be complexed to lipids, in particular to cationic lipids. Preferred fatty acids and esters, pharmaceutically acceptable salts thereof, and their uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. In one embodiment, Oral formulations are those in which oligonucleotides are administered in conjunction with one or more penetration enhancers surfactants and chelators. Surfactants include fatty acids and/or esters or salts thereof, bile acids and/or salts thereof. Preferred bile acids/salts and fatty acids and their uses are further described in U.S. Pat. No. 6,287,860, which is incorporated herein in its entirety. Also preferred are combinations of penetration enhancers, for example, fatty acids/salts in combination with bile acids/salts. One combination is the sodium salt of lauric acid, capric acid and UDCA. Further penetration enhancers include polyoxyethylene-9-lauryl ether, polyoxyethylene-20-cetyl ether. Oligonucleotides may be delivered orally, in granular form including sprayed dried particles, or complexed to form micro or nanoparticles. Oligonucleotide complexing agents and their uses are further described in U.S. Pat. No. 6,287,860, which is incorporated herein in its entirety. Oral formulations for oligonucleotides and their preparation are described in detail in U.S. application Ser. No. 09/108,673 (filed Jul. 1, 1998), 09/315,298 (filed May 20, 1999) and 10/071,822, filed Feb. 8, 2002, each of which is incorporated herein by reference in their entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Certain embodiments provide pharmaceutical compositions containing one or more oligomeric compounds and one or more other chemotherapeutic agents which function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compounds described herein, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions. Combinations of antisense compounds and other non-antisense drugs are also within the scope of this invention. Two or more combined compounds may be used together or sequentially.

In another related embodiment, compositions may contain one or more antisense compounds, particularly oligonucleotides, targeted to a first nucleic acid and one or more additional antisense compounds targeted to a second nucleic acid target. Alternatively, compositions may contain two or more antisense compounds targeted to different regions of the same nucleic acid target. Numerous examples of antisense compounds are known in the art. Two or more combined compounds may be used together or sequentially.

H. Dosing

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

While the present invention has been described with specificity in accordance with certain of its preferred embodiments, the following examples serve only to illustrate the invention and are not intended to limit the same. Each of the references, GenBank accession numbers, and the like recited in the present application is incorporated herein by reference in its entirety.

### EXAMPLES

### Example 1

Synthesis of Nucleoside Phosphoramidites

The following compounds, including amidites and their intermediates were prepared as described in U.S. Pat. No. 6,426,220 and published PCT WO 02/36743; 5′-O-Dimethoxytrityl-thymidine intermediate for 5-methyl dC amidite, 5′-O-Dimethoxytrityl-2′-deoxy-5-methylcytidine intermediate for 5-methyl-dC amidite, 5¹-O-Dimethoxytrityl-2′-deoxy-N4-benzoyl-5-methylcytidine penultimate intermediate for 5-methyl dC amidite, [5′-O-(4,4′-Dimethoxytriphenylmethyl)-2′-deoxy-N⁴-benzoyl-5-methylcytidin-3′-O-yl]-2-cyanoethyl-N,N-diisopropylphosphoramidite (5-methyl dC amidite), 2′-Fluorodeoxyadenosine, 2′-Fluorodeoxyguanosine, 2′-Fluorouridine, 2′-Fluorodeoxycytidine, 2′-O-(2-Methoxyethyl) modified amidites, 2′-O-(2-methoxyethyl)-5-methyluridine intermediate, 5′-O-DMT-2′-O-(2-methoxyethyl)-5-methyluridine penultimate intermediate, [5′-O-(4,4′-Dimethoxytriphenylmethyl)-2′-O-(2-methoxyethyl)-5-methyluridin-3′-O-yl]-2-cyanoethyl-N,N-diisopropylphosphoramidite (MOE T amidite), 51-O-Dimethoxytrityl-2′-O-(2-methoxyethyl)-5-methylcytidine intermediate, 5′-O-dimethoxytrityl-2′-O-(2-methoxyethyl)-N⁴-benzoyl-5-methyl-cytidine penultimate intermediate, [5′-O-(4,4′-Dimethoxytriphenylmethyl)-2′-O-(2-methoxyethyl)-N⁴-benzoyl-5-methylcytidin-3′-O-yl]-2-cyanoethyl-N,N-diisopropylphosphoramidite (MOE 5-Me-C amidite), [5′-O-(4,4′-Dimethoxytriphenylmethyl)-2′-O-(2-methoxyethyl)-N⁶-benzoyladenosin-3′-O-yl]-2-cyanoethyl-N,N-diisopropylphosphoramidite (MOE A amdite), [5′-O-(4,4′-Dimethoxytriphenylmethyl)-2′-O-(2-methoxyethyl)-N⁴-isobutyrylguanosin-3′-O-yl]-2-cyanoethyl-N,N-diisopropylphosphoramidite (MOE G amidite), 2′-O-(Aminooxyethyl) nucleoside amidites and 2′-O-(dimethylaminooxyethyl) nucleoside amidites, 2′-(Dimethylaminooxyethoxy) nucleoside amidites, 5′-O-tert-Butyldiphenylsilyl-O²-2′-anhydro-5-methyluridine, 5′-O-tert-Butyldiphenylsilyl-2′-O-(2-hydroxyethyl)-5-methyluridine, 2′-O-([2-phthalimidoxy)ethyl]-5′-t-butyldiphenylsilyl-5-methyluridine 5′-O-tert-butyldiphenylsilyl-2′-O-[(2-formadoximinooxy)ethyl]-5-methyluridine, 5′-O-tert-Butyldiphenylsilyl-2′-O-[N,N dimethylaminooxyethyl]-5-methyluridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-[(2-cyanoethyl)-N,N-diisopropylphosphoramidite], 2′-(Aminooxyethoxy) nucleoside amidites, N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite], 2′-dimethylaminoethoxyethoxy (2′-DMAEOE) nucleoside amidites, 2′-O-[2(2-N,N-dimethylaminoethoxy)ethyl]-5-methyl uridine, 5′-O-dimethoxytrityl-2′-O-[2(2-N,N-dimethylaminoethoxy)-ethyl)]-5-methyl uridine and 5′-O-Dimethoxytrityl-2′-O-[2(2-N,N-dimethylaminoethoxy)-ethyl)]-5-methyl uridine-3′-O-(cyanoethyl-N,N-diisopropyl)phosphoramidite.

### Example 2

Oligonucleotide and Oligonucleoside Synthesis

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH₄OAc solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. Nos. 5,610,289 or 5,625,050, herein incorporated by reference.

Phosphoramidite oligonucleotides are prepared as described in U.S. Pat. Nos. 5,256,775 or 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

Oligonucleosides: Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 3

RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′- groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and oligonucleotide synthesis. However, after oligonucleotide synthesis the oligonucleotide is treated with methylamine which not only cleaves the oligonucleotide from the solid support but also removes the acetyl groups from the orthoesters. The resulting 2-ethyl-hydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand,. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

RNA antisense compounds (RNA oligonucleotides) of the present invention can be synthesized by the methods herein or purchased from Dharmacon Research, Inc (Lafayette, Colo.). Once synthesized, complementary RNA antisense compounds can then be annealed by methods known in the art to form double stranded (duplexed) antisense compounds. For example, duplexes can be formed by combining 30 μl of each of the complementary strands of RNA oligonucleotides (50 uM RNA oligonucleotide solution) and 15 μl of 5× annealing buffer (100 mM potassium acetate, 30 mM HEPES-KOH pH 7.4, 2 mM magnesium acetate) followed by heating for 1 minute at 90° C., then 1 hour at 37° C. The resulting duplexed antisense compounds can be used in kits, assays, screens, or other methods to investigate the role of a target nucleic acid, or for diagnostic or therapeutic purposes.

### Example 4

Synthesis of Chimeric Compounds

Chimeric oligonucleotides, oligonucleosides or mixed oligonucleotides/oligonucleosides can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

[2′-O-Me]--[2′-deoxy]--[2′-O-Me] Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 394, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH₄OH) for 12-16 hr at 55° C. The deprotected oligo is then recovered by an appropriate method (precipitation, column chromatography, volume reduced in vacuo and analyzed spetrophotometrically for yield and for purity by capillary electrophoresis and by mass spectrometry.

[2′-O-(2-Methoxyethyl)]--[2′-deoxy]--[2′-O-(Methoxyhethyl)] Chimeric Phosphorothioate Oligonucleotides

[2′-O-(2-methoxyethyl)]--[2′-deoxy]--[-2′-O-(methoxyethyl)] chimeric phosphorothioate oligonucleotides were prepared as per the procedure above for the 2′-O-methyl chimeric oligonucleotide, with the substitution of 2′-O-(methoxyethyl) amidites for the 2′-O-methyl amidites.

[2′-O-(2-Methoxyethyl)Phosphodiester]--[2′-deoxy Phosphorothioate]--[2′-O-(2-Methoxyethyl) Phosphodiester] Chimeric Oligonucleotides

[2′-O-(2-methoxyethyl phosphodiester]--[2′-deoxy phosphorothioate]--[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U.S. Pat. No. 5,623,065, herein incorporated by reference.

### Example 5

Design and Screening of Duplexed Antisense Compounds Targeting DC-SIGN

In accordance with the present invention, a series of nucleic acid duplexes comprising the antisense compounds of the present invention and their complements can be designed to target DC-SIGN. The nucleobase sequence of the antisense strand of the duplex comprises at least an 8-nucleobase portion of an oligonucleotide in Table 1. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG and having a two-nucleobase overhang of deoxythymidine(dT) would have the following structure:

| | |
|---|---|
| cgagaggcggacgggaccgTT | Antisense Strand |
| \|\|\|\|\|\|\|\|\|\|\|\|\|\|\|\|\|\|\| | |
| TTgctctccgcctgccctggc | Complement |

In another embodiment, a duplex comprising an antisense strand having the same sequence CGAGAGGCGGACGGGACCG may be prepared with blunt ends (no single stranded overhang) as shown:

| | |
|---|---|
| cgagaggcggacgggaccg | Antisense Strand |
| \|\|\|\|\|\|\|\|\|\|\|\|\|\|\|\|\|\|\| | |
| gctctccgcctgccctggc | Complement |

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 uM. Once diluted, 30 uL of each strand is combined with 15uL of a 5× solution of annealing buffer. The final concentration of said buffer is 100 mM potassium acetate, 30 mM HEPES-KOH pH 7.4, and 2mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed antisense compounds are evaluated for their ability to modulate DC-SIGN expression.

When cells reached 80% confluency, they are treated with duplexed antisense compounds. For cells grown in 96-well plates, wells are washed once with 200 pL OPTI-MEM-1 reduced-serum medium (Gibco BRL) and then treated with 130 uL of OPTI-MEM-1 containing 12 μg/mL LIPOFECTIN (Gibco BRL) and the desired duplex antisense compound at a final concentration of 200 nM. After 5 hours of treatment, the medium is replaced with fresh medium. Cells are harvested 16 hours after treatment, at which time RNA is isolated and target reduction measured by RT-PCR.

### Example 6

Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the −16 amu product (+/−32+/−48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 7

Oligonucleotide Synthesis—96 Well Plate Format

Oligonucleotides were synthesized via solid phase P(III) phosphoramidite chemistry on an automated synthesizer capable of assembling 96 sequences simultaneously in a 96-well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 8

Oligonucleotide Analysis—96-Well Plate Format The concentration of oligonucleotide in each well was assessed by dilution of samples and UV absorption spectroscopy. The full-length integrity of the individual products was evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 9

Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

T-24 Cells:

The human transitional cell bladder carcinoma cell line T-24 was obtained from the American Type Culture Collection (ATCC) (Manassas, Va.). T-24 cells were routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 96-well plates (Falcon-Primaria #353872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

A549 Cells:

The human lung carcinoma cell line A549 was obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

NHDF Cells:

Human neonatal dermal fibroblast (NHDF) were obtained from the Clonetics Corporation (Walkersville, Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

HEK Cells:

Human embryonic keratinocytes (HEK) were obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier.

THP-1 Cells:

The acute monocytic leukemia cell line, THP-1, was obtained from the American Type Culture Collection (ATCC) (Manassas, Va.). The cells are routinely cultured at 37° C. in ATCC medium: RPMI 1640 medium with 2 mM L-glutamine adjusted to contain 1.5 g/L sodium bicarbonate, 4.5 g/L glucose, 10 mM HEPES and 1.0 mM sodium pyruvate and supplemented with 20% fetal bovine serum.

Dendritic Cells Differentiated from Bone Marrow:

Mouse dendritic cells were differentiated from the bone marrow of ˜4-month old, female BALB/C mice purchased from Charles River Laboratories. Mouse bone marrow was flushed from tibia and femur bones using RPMI 1640 media (Gibco #11875-093) containing 5% heat inactivated Fetal Bovine Serum, 10 mM HEPES, 20 μg/mL gentamycin, and 50 μM β-mercaptoethanol. Dendritic cells were differentiated from the bone marrow in the same media containing 20 ng/mL granulocyte macrophage-colony stimulating factor (GM-CSF) (R&D systems #415-ML). 150,000 dendritic cells were plated into each well of a 24 well plate (Falcon Primaria #353847).

Treatment with Antisense Compounds:

When cells reached 65-75% confluency, they were treated with oligonucleotide. Unless otherwise noted, cells were transfected with LIPOFECTIN™ (Invitrogen Corporation, Carlsbad, Calif.). For cells grown in 96-well plates, wells were washed once with 100 μL OPTI-MEM™-1 reduced-serum medium (Invitrogen Corporation, Carlsbad, Calif.) and then treated with 130 μL of OPTI-MEM™-1 containing 3.75 μg/mL LIPOFECTIN™ and the desired concentration of oligonucleotide. Cells are treated and data are obtained in triplicate. After 4-7 hours of treatment at 37° C., the medium was replaced with fresh medium. Cells were harvested 16-24 hours after oligonucleotide treatment.

THP-1 cells are suspension cells and are therefore treated via electroporation. When the cell count reaches 2×10⁷ cells/mL, 90 ul volumes of cell suspension are treated with 20 ul of 50 uM oligonucleotide (total concentration of 10 uM). This mixture is then transferred to a 1 mm gap cuvette. Then, 75 V is applied to the cuvette for 6 msec, after which, 800 ul growth media (RPMI) is added and mixed in the cuvette. 800 ul of this mixture is then transferred to a 24-well plate already containing 1 mL of growth media (RPMI) in each well (to give a total volume of 1.9 mL). After incubation overnight at 5% CO², 800 ul of the mixture is transferred in duplicate from the 24-well plate to a 96-well deep well block. After centrifugation for 10 minutes at 2500 rpm, the media is decanted and cells are lysed in 150 ul of RLT buffer (Qiagen, Valencia, Calif.). RNA harvested from these mixtures is used in the standard real time PCR assay.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TCCGTCATCGCTCCTCAGGG, SEQ ID NO: 1) which is targeted to human H-ras, or ISIS 18078, (GTGCGCGCGAGCCCGAAATC, SEQ ID NO: 2) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligonucleotide is ISIS 15770, ATGCATTCTGCCCCCAAGGA, SEQ ID NO: 3, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-H-ras (for ISIS 13920), JNK2 (for ISIS 18078) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of c-H-ras, JNK2 or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 10 nM to 300 nM.

### Example 10

Analysis of Oligonucleotide Inhibition of DC-SIGN Expression

Antisense modulation of DC-SIGN expression can be assayed in a variety of ways known in the art. For example, DC-SIGN mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. The preferred method of RNA analysis of the present invention is the use of total cellular RNA as described in other examples herein. Methods of RNA isolation are well known in the art. Northern blot analysis is also routine in the art. Real-time quantitative (PCR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of DC-SIGN can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to DC-SIGN can be identified and obtained from a variety of sources, such as the MSRS catalog of antibodies (Aerie Corporation, Birmingham, Mich.), or can be prepared via conventional monoclonal or polyclonal antibody generation methods well known in the art.

### Example 11

Design of Phenotypic Assays for the Use of DC-SIGN Inhibitors

Phenotyopic Assays

Once DC-SIGN inhibitors have been identified by the methods disclosed herein, the compounds are further investigated in one or more phenotypic assays, each having measurable endpoints predictive of efficacy in the treatment of a particular disease state or condition. Phenotypic assays, kits and reagents for their use are well known to those skilled in the art and are herein used to investigate the role and/or association of DC-SIGN in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with DC-SIGN inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Analysis of the genotype of the cell (measurement of the expression of one or more of the genes of the cell) after treatment is also used as an indicator of the efficacy or potency of the DC-SIGN inhibitors. Hallmark genes, or those genes suspected to be associated with a specific disease state, condition, or phenotype, are measured in both treated and untreated cells.

### Example 12

RNA Isolation

Poly(A)+ mRNA Isolation

Poly(A)+ mRNA was isolated according to Miura et al., (Clin. Chem., 1996, 42, 1758-1764). Other methods for poly(A)+ mRNA isolation are routine in the art. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

Total RNA Isolation

Total RNA was isolated using an RNEASY 96™ kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 1 minute. 500 μL of Buffer RW1 was added to each well of the RNEASY 96™ plate and incubated for 15 minutes and the vacuum was again applied for 1 minute. An additional 500 μL of Buffer RW1 was added to each well of the RNEASY 96™ plate and the vacuum was applied for 2 minutes. 1 mL of Buffer RPE was then added to each well of the RNEASY 96™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 13

Real-time Quantitative PCR Analysis of DC-SIGN mRNA Levels

Quantitation of DC-SIGN mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5×PCR buffer minus MgCl₂, 6.6 mM MgCl₂, 375 μM each of DATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

Probes and primers to human DC-SIGN were designed to hybridize to a human DC-SIGN sequence, using published sequence information (GenBank accession number NM_{—}021155.2, incorporated herein as SEQ ID NO: 4). For human DC-SIGN the PCR primers were:

forward primer: CATGCCTCACTACCCTCTGTATATAAAA (SEQ ID NO: 5)

reverse primer: ACACAGGAGTCTGGCCCAAA (SEQ ID NO: 6) and the PCR probe was: FAM-CCCCAGCCTCCAACTTGGAGAGACAG-TAMRA (SEQ ID NO: 7) where FAM is the fluorescent dye and TAMRA is the quencher dye. For human GAPDH the PCR primers were:

forward primer: GAAGGTGAAGGTCGGAGTC(SEQ ID NO: 8)

reverse primer: GAAGATGGTGATGGGATTTC (SEQ ID NO: 9) and the PCR probe was: 5′ JOE-CAAGCTTCCCGTTCTCAGCC-TAMRA 3′ (SEQ ID NO: 10) where JOE is the fluorescent reporter dye and TAMRA is the quencher dye.

Probes and primers to mouse DC-SIGN were designed to hybridize to a mouse DC-SIGN sequence, using published sequence information (GenBank accession number XM_{—}134025.1, incorporated herein as SEQ ID NO: 11). For mouse DC-SIGN the PCR primers were:

forward primer: CCTCTGGATGAGGAACTGCTG (SEQ ID NO: 12)

reverse primer: TGGAAGCCAAAGCCTTTGA (SEQ ID NO: 13) and the PCR probe was: FAM-CATCCAGCCACACCAGGCACTCC-TAMRA (SEQ ID NO: 14) where FAM is the fluorescent reporter dye and TAMRA is the quencher dye. For mouse GAPDH the PCR primers were:

forward primer: GGCAAATTCAACGGCACAGT(SEQ ID NO: 15)

reverse primer: GGGTCTCGCTCCTGGAAGAT(SEQ ID NO: 16) and the PCR probe was: 5′ JOE-AAGGCCGAGAATGGGAAGCTTGTCATC-TAMRA 3′ (SEQ ID NO: 17) where JOE is the fluorescent reporter dye and TAMRA is the quencher dye.

### Example 14

Northern Blot Analysis of DC-SIGN mRNA Levels

Eighteen hours after antisense treatment, cell monolayers were washed twice with cold PBS and lysed in 1 mL RNAZOL™ (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, OH). RNA was transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect human DC-SIGN, a human DC-SIGN specific probe was prepared by PCR using the forward primer CATGCCTCACTACCCTCTGTATATAAAA (SEQ ID NO: 5) and the reverse primer ACACAGGAGTCTGGCCCAAA (SEQ ID NO: 6). To normalize for variations in loading and transfer efficiency membranes were stripped and probed for human glyceraldehyde-3-phosphate dehydrogenase (GAPDH) RNA (Clontech, Palo Alto, Calif.).

To detect mouse DC-SIGN, a mouse DC-SIGN specific probe was prepared by PCR using the forward primer CCTCTGGATGAGGAACTGCTG (SEQ ID NO: 12) and the reverse primer TGGAAGCCAAAGCCTTTGA (SEQ ID NO: 13). To normalize for variations in loading and transfer efficiency membranes were stripped and probed for mouse glyceraldehyde-3-phosphate dehydrogenase (GAPDH) RNA (Clontech, Palo Alto, Calif.).

Hybridized membranes were visualized and quantitated using a PHOSPHORIMAGER™ and IMAGEQUANT™ Software V3.3 (Molecular Dynamics, Sunnyvale, Calif.). Data was normalized to GAPDH levels in untreated controls.

### Example 15

Antisense Inhibition of Human DC-SIGN Expression by Chimeric Phosphorothioate Oligonucleotides having 2′-MOE Wings and a Deoxy Gap

In accordance with the present invention, a series of antisense compounds was designed to target different regions of the human DC-SIGN RNA, using published sequences (GenBank accession number NM_{—}021155.2, incorporated herein as SEQ ID NO: 4, GenBank accession number M98457.1, incorporated herein as SEQ ID NO: 18, GenBank accession number AY042222.1, incorporated herein as SEQ ID NO: 19, GenBank accession number AY042223.1, incorporated herein as SEQ ID NO: 20, GenBank accession number AY042224.1, incorporated herein as SEQ ID NO: 21, GenBank accession number AY042225.1, incorporated herein as SEQ ID NO: 22, GenBank accession number AY042226.1, incorporated herein as SEQ ID NO: 23, GenBank accession number AY042229.1, incorporated herein as SEQ ID NO: 24, GenBank accession number AY042232.1, incorporated herein as SEQ ID NO: 25, GenBank accession number AK057212.1, incorporated herein as SEQ ID NO: 26, and nucleotides 408478 to 416828 of the sequence with GenBank accession number NT_{—}077812.1, the complement of which is incorporated herein as SEQ ID NO: 27). The compounds are shown in Table 1. “Target site” indicates the first (5′-most) nucleotide number on the particular target sequence to which the compound binds. All compounds in Table 1 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on human DC-SIGN mRNA levels by quantitative real-time PCR as described in other examples herein. Data are averages from two experiments in which THP-1 cells were treated with 10 μM of the antisense oligonucleotides of the present invention. The positive control for each datapoint is identified in the table by sequence ID number. The positive control oligonucleotide is ISIS 13560 (TCCCGCCTGTGACATGCATT, SEQ ID NO: 370) which is targeted to human raf kinase C. If present, “N.D.” indicates “no data”.

TABLE 1

Inhibition of human DC-SIGN mRNA levels by chimeric
phosphorothioate oligonucleotides having 2′-MOE wings and a
deoxy gap

| | | | | | | | |
|---|---|---|---|---|---|---|---|
| | | TARGET | | | | | |
| | | SEQ ID | TARGET | | % | SEQ | CONTROL |
| ISIS # | REGION | NO | SITE | SEQUENCE | INHIB | ID NO | SEQ ID NO |

| | | | | | | | |
|---|---|---|---|---|---|---|---|
| 344026 | 5′UTR | 18 | 10 | tcccccagtgtccagaactc | 11 | 28 | 370 |
| 344027 | Start | 4 | 1 | gagtcactcatgtcacccca | 87 | 29 | 370 |
| | Codon | | | | | | |
| 344028 | Coding | 4 | 48 | gctgttcctcctccaggagg | 0 | 30 | 370 |
| 344029 | Coding | 4 | 77 | tcgagtctgtcggaatccaa | 28 | 31 | 370 |
| 344030 | Coding | 4 | 100 | caccctgctaagctcttgta | 62 | 32 | 370 |
| 344031 | Coding | 4 | 166 | tggacaaggagcccagccaa | 51 | 33 | 370 |
| 344032 | Coding | 4 | 486 | tcttagatttctctggaagc | 78 | 34 | 370 |
| 344033 | Coding | 4 | 1012 | tgcttgaagctgggcaacag | 4 | 35 | 370 |
| 344034 | Coding | 4 | 1017 | aatactgcttgaagctgggc | 7 | 36 | 370 |
| 344035 | Coding | 4 | 1117 | atccagaatttggcaagatt | 57 | 37 | 370 |
| 344036 | 3′UTR | 4 | 1240 | gaaggaactgtagcttaaaa | 41 | 38 | 370 |
| 344037 | 3′UTR | 4 | 1291 | aatctgacaaagaacagtcc | 5 | 39 | 370 |
| 344038 | 3′UTR | 4 | 1307 | cttctaaaggaggaagaatc | 0 | 40 | 370 |
| 344039 | 3′UTR | 4 | 1316 | ggacccagccttctaaagga | 76 | 41 | 370 |
| 344040 | 3′UTR | 4 | 1441 | tcttggaacacaagtccacc | 53 | 42 | 370 |
| 344041 | 3′UTR | 4 | 1485 | ggcaacccaaatatcctaat | 84 | 43 | 370 |
| 344042 | 3′UTR | 4 | 1590 | gtaagtccccgatcaaaggg | 41 | 44 | 370 |
| 344043 | 3′UTR | 4 | 2176 | cctctagatcccaaagtcgt | 73 | 45 | 370 |
| 344044 | 3′UTR | 4 | 2185 | gccaaagctcctctagatcc | 49 | 46 | 370 |
| 344045 | 3′UTR | 4 | 2201 | agccgtcacagccggagcca | 65 | 47 | 370 |
| 344046 | 3′UTR | 4 | 2224 | gcagccgcagtgagaacggc | 67 | 48 | 370 |
| 344047 | 3′UTR | 4 | 2291 | tcaaagagccaggagaggca | 33 | 49 | 370 |
| 344048 | 3′UTR | 4 | 2369 | gagaaccctagtccagacca | 87 | 50 | 370 |
| 344049 | 3′UTR | 4 | 2395 | gatgaatactacaggctgcg | 47 | 51 | 370 |
| 344050 | 3′UTR | 4 | 2405 | gggccaccacgatgaatact | 81 | 52 | 370 |
| 344051 | 3′UTR | 4 | 2459 | gagacctctgcttgtctcct | 69 | 53 | 370 |
| 344052 | 3′UTR | 4 | 2473 | tgagaaatccaatagagacc | 63 | 54 | 370 |
| 344053 | 3′UTR | 4 | 2485 | ttcttcaggtgttgagaaat | 29 | 55 | 370 |
| 344054 | 3′UTR | 4 | 2531 | ggtttatttgaaatacaaca | 67 | 56 | 370 |
| 344055 | 3′UTR | 4 | 2580 | gcttcagtgtgaattctgcc | 85 | 57 | 370 |
| 344056 | 3′UTR | 4 | 2629 | agcagctctttctctgttta | 65 | 58 | 370 |
| 344057 | 3′UTR | 4 | 2664 | atatatgttcagtaccagtc | 73 | 59 | 370 |
| 344058 | 3′UTR | 4 | 2709 | agttcaaggtcagttgcaac | 63 | 60 | 370 |
| 344059 | 3′UTR | 4 | 2720 | gagagtgattcagttcaagg | 60 | 61 | 370 |
| 344060 | 3′UTR | 4 | 3077 | cccgagttcaagaacgtggg | 83 | 62 | 370 |
| 344061 | 3′UTR | 4 | 3093 | ctgtgaggatgtgctgcccg | 79 | 63 | 370 |
| 344062 | 3′UTR | 4 | 3108 | acagttcctagatttctgtg | 66 | 64 | 370 |
| 344063 | 3′UTR | 4 | 3170 | tgcaggagggatgactatga | 33 | 65 | 370 |
| 344064 | 3′UTR | 4 | 3202 | aaagcatgtttacagtgttt | 96 | 66 | 370 |
| 344065 | 3′UTR | 4 | 3213 | ccttcttattaaaagcatgt | 83 | 67 | 370 |
| 344066 | 3′UTR | 4 | 3246 | atgttcacatgatttcccaa | 73 | 68 | 370 |
| 344067 | 3′UTR | 4 | 3268 | gagcttgcagatttggagat | 32 | 69 | 370 |
| 344068 | 3′UTR | 4 | 3283 | tctacagtaaaacaggagct | 88 | 70 | 370 |
| 344069 | 3′UTR | 4 | 3593 | aaagattacatgaggagtgg | 0 | 71 | 370 |
| 344070 | 3′UTR | 4 | 3664 | actctcacagaaagaggagg | 64 | 72 | 370 |
| 344071 | 3′UTR | 4 | 3681 | ccactgagggactcaggact | 80 | 73 | 370 |
| 344072 | 3′UTR | 4 | 3731 | gggccgccctgaagaaggag | 25 | 74 | 370 |
| 344073 | 3′UTR | 4 | 3787 | gatggctgcgcccgaaaagg | 23 | 75 | 370 |
| 344074 | 3′UTR | 4 | 3794 | ccggcaagatggctgcgccc | 71 | 76 | 370 |
| 344075 | 3′UTR | 4 | 3863 | gggctgaatctgcggttaca | 86 | 77 | 370 |
| 344076 | 3′UTR | 4 | 3974 | tttgcacgcttcgttcatct | 87 | 78 | 370 |
| 344077 | 3′UTR | 4 | 4008 | gttcctcagtctgagcactt | 82 | 79 | 370 |
| 344078 | 3′UTR | 4 | 4017 | tcagtccctgttcctcagtc | 64 | 80 | 370 |
| 344079 | 3′UTR | 4 | 4028 | cacttcttaactcagtccct | 41 | 81 | 370 |
| 344080 | 3′UTR | 4 | 4090 | gatctgactggatcacgcca | 81 | 82 | 370 |
| 344081 | 3′UTR | 4 | 4101 | tcaggaggcttgatctgact | 96 | 83 | 370 |
| 344086 | Coding | 19 | 891 | ttctggaagactgctcctca | 0 | 84 | 370 |
| 344087 | Coding | 20 | 458 | gcgttccactgcagccttca | 72 | 85 | 370 |
| 344088 | Coding | 21 | 200 | tctggattgttcctgactta | 56 | 86 | 370 |
| 344089 | Coding | 22 | 97 | accttggacactgctaagct | 0 | 87 | 370 |
| 344090 | Coding | 23 | 37 | accttggacaccaggaggcc | 0 | 88 | 370 |
| 344091 | Coding | 24 | 139 | gctgttcctcctctgaatgg | 0 | 89 | 370 |
| 344092 | Coding | 25 | 919 | ctgcttgaagctgctcctca | 34 | 90 | 370 |
| 344093 | 3′UTR | 26 | 1308 | tgttcacatgatttctgtga | 14 | 91 | 370 |
| 344094 | Intron:Exon | 27 | 572 | tgttcctcctctgaatggat | 19 | 92 | 370 |
| | Junction | | | | | | |
| 344095 | Exon:Intron | 27 | 632 | tcctctctacctgctaagct | 26 | 93 | 370 |
| | Junction | | | | | | |
| 344096 | Exon:Intron | 27 | 1478 | tgcccctgaccttggacaag | 5 | 94 | 370 |
| | Junction | | | | | | |
| 344097 | Exon:Intron | 27 | 1882 | gcctggattgttcctgactt | 49 | 95 | 370 |
| | Junction | | | | | | |
| 344098 | Intron | 27 | 2016 | agatttctctggaagctcac | 89 | 96 | 370 |
| 344099 | Intron | 27 | 2109 | ctcctggtagatctcctgca | 83 | 97 | 370 |
| 344100 | Exon:Intron | 27 | 2144 | ggaagctcacccactgcagc | 96 | 98 | 370 |
| | Junction | | | | | | |
| 344101 | Exon:Intron | 27 | 2997 | ccaggtgtacctgctcctca | 45 | 99 | 370 |
| | Junction | | | | | | |
| 344102 | Intron | 27 | 3343 | tgagtgacatcacaacgaca | 11 | 100 | 370 |
| 344103 | Intron:Exon | 27 | 3736 | ttctggaagactgcagctgt | 39 | 101 | 370 |
| | Junction | | | | | | |
| 344104 | Coding | 4 | 25 | agctgctgcagtcttggttc | 16 | 102 | 370 |
| 344105 | Coding | 4 | 105 | caagacaccctgctaagctc | 0 | 103 | 370 |
| 344106 | Coding | 4 | 110 | atggccaagacaccctgcta | 16 | 104 | 370 |
| 344107 | Coding | 4 | 134 | gaggagttgcagcaccaggg | 49 | 105 | 370 |
| 344108 | Coding | 4 | 139 | aaggagaggagttgcagcac | 37 | 106 | 370 |
| 344109 | Coding | 4 | 149 | caagagcgtgaaggagagga | 0 | 107 | 370 |
| 344110 | Coding | 4 | 154 | ccagccaagagcgtgaagga | 15 | 108 | 370 |
| 344111 | Coding | 4 | 159 | ggagcccagccaagagcgtg | 19 | 109 | 370 |
| 344112 | Coding | 4 | 171 | acacttggacaaggagccca | 32 | 110 | 370 |
| 344113 | Coding | 4 | 275 | ggatttctctgagagctcac | 67 | 111 | 370 |
| 344114 | Coding | 4 | 284 | ctgcagcttggatttctctg | 51 | 112 | 370 |
| 344115 | Coding | 4 | 289 | atctcctgcagcttggattt | 89 | 113 | 370 |
| 344116 | Coding | 4 | 294 | ggtagatctcctgcagcttg | 96 | 114 | 370 |
| 344117 | Coding | 4 | 304 | gtcagctcctggtagatctc | 79 | 115 | 370 |
| 344118 | Coding | 4 | 317 | agccttcagctgggtcagct | 79 | 116 | 370 |
| 344119 | Coding | 4 | 322 | actgcagccttcagctgggt | 76 | 117 | 370 |
| 344120 | Coding | 4 | 328 | tcacccactgcagccttcag | 83 | 118 | 370 |
| 344121 | Coding | 4 | 333 | gaagctcacccactgcagcc | 95 | 119 | 370 |
| 344122 | Coding | 4 | 338 | ctctggaagctcacccactg | 87 | 120 | 370 |
| 344123 | Coding | 4 | 343 | gatttctctggaagctcacc | 93 | 121 | 370 |
| 344124 | Coding | 4 | 364 | tggtagatctcctgcagctt | 57 | 122 | 370 |
| 344125 | Coding | 4 | 369 | gctcctggtagatctcctgc | 91 | 123 | 370 |
| 344126 | Coding | 4 | 374 | ggtcagctcctggtagatct | 87 | 124 | 370 |
| 344127 | Coding | 4 | 379 | agccgggtcagctcctggta | 83 | 125 | 370 |
| 344128 | Coding | 4 | 384 | ccttcagccgggtcagctcc | 39 | 126 | 370 |
| 344129 | Coding | 4 | 389 | tgcagccttcagccgggtca | 45 | 127 | 370 |
| 344130 | Coding | 4 | 448 | agccaggtcagctcctggta | 86 | 128 | 370 |
| 344131 | Coding | 4 | 453 | ccttcagccaggtcagctcc | 53 | 129 | 370 |
| 344132 | Coding | 4 | 458 | tgcagccttcagccaggtca | 82 | 130 | 370 |
| 344133 | Coding | 4 | 491 | ctgcatcttagatttctctg | 82 | 131 | 370 |
| 344134 | Coding | 4 | 496 | atctcctgcatcttagattt | 63 | 132 | 370 |
| 344135 | Coding | 4 | 501 | ggtagatctcctgcatctta | 88 | 133 | 370 |
| 344136 | Coding | 4 | 571 | tggtagatctcctgctgctt | 94 | 134 | 370 |
| 344137 | Coding | 4 | 741 | gttccactgcagccttcagc | 75 | 135 | 370 |
| 344138 | Coding | 4 | 787 | tttccttggaagaatgtcca | 30 | 136 | 370 |
| 344139 | Coding | 4 | 792 | aacagtttccttggaagaat | 18 | 137 | 370 |
| 344140 | Coding | 4 | 814 | cgctgggagttagacatgaa | 24 | 138 | 370 |
| 344141 | Coding | 4 | 819 | agttccgctgggagttagac | 0 | 139 | 370 |
| 344142 | Coding | 4 | 824 | gtgccagttccgctgggagt | 49 | 140 | 370 |
| 344143 | Coding | 4 | 829 | gagtcgtgccagttccgctg | 59 | 141 | 370 |
| 344144 | Coding | 4 | 834 | tgatggagtcgtgccagttc | 33 | 142 | 370 |
| 344145 | Coding | 4 | 868 | acgacgagctgggcccccac | 69 | 143 | 370 |
| 344146 | Coding | 4 | 873 | tgattacgacgagctgggcc | 48 | 144 | 370 |
| 344147 | Coding | 4 | 878 | acttttgattacgacgagct | 5 | 145 | 370 |
| 344148 | Coding | 4 | 883 | tcagcacttttgattacgac | 46 | 146 | 370 |
| 344149 | Coding | 4 | 888 | gctcctcagcacttttgatt | 39 | 147 | 370 |
| 344150 | Coding | 4 | 893 | gttctgctcctcagcacttt | 54 | 148 | 370 |
| 344151 | Coding | 4 | 898 | aggaagttctgctcctcagc | 44 | 149 | 370 |
| 344152 | Coding | 4 | 919 | cttctggaagactgcagctg | 38 | 150 | 370 |
| 344153 | Coding | 4 | 924 | ggttacttctggaagactgc | 69 | 151 | 370 |
| 344154 | Coding | 4 | 929 | gaagcggttacttctggaag | 51 | 152 | 370 |
| 344155 | Coding | 4 | 934 | caggtgaagcggttacttct | 86 | 153 | 370 |
| 344156 | Coding | 4 | 939 | ccatccaggtgaagcggtta | 68 | 154 | 370 |
| 344157 | Coding | 4 | 944 | aagtcccatccaggtgaagc | 41 | 155 | 370 |
| 344158 | Coding | 4 | 949 | tctgaaagtcccatccaggt | 84 | 156 | 370 |
| 344159 | Coding | 4 | 1003 | ctgggcaacagaggtgagcc | 0 | 157 | 370 |
| 344160 | Coding | 4 | 1008 | tgaagctgggcaacagaggt | 9 | 158 | 370 |
| 344161 | Coding | 4 | 1013 | ctgcttgaagctgggcaaca | 24 | 159 | 370 |
| 344162 | Coding | 4 | 1018 | caatactgcttgaagctggg | 66 | 160 | 370 |
| 344163 | Coding | 4 | 1023 | tgttccaatactgcttgaag | 63 | 161 | 370 |
| 344164 | Coding | 4 | 1078 | ccattgccactaaattccgc | 52 | 162 | 370 |
| 344165 | Coding | 4 | 1083 | tccagccattgccactaaat | 83 | 163 | 370 |
| 344166 | Coding | 4 | 1126 | tttttgcagatccagaattt | 0 | 164 | 370 |
| 344167 | 3′UTR | 4 | 1269 | gagattttgtgaaggtcgaa | 85 | 165 | 370 |
| 344168 | 3′UTR | 4 | 1329 | gaaggacagaatgggaccca | 86 | 166 | 370 |
| 344169 | 3′UTR | 4 | 1377 | gggccagaaaaacaagctct | 78 | 167 | 370 |
| 344170 | 3′UTR | 4 | 1474 | tatcctaatctccaaaagga | 53 | 168 | 370 |
| 344171 | 3′UTR | 4 | 2119 | agagtcccaaatcccagtaa | 38 | 169 | 370 |
| 344172 | 3′UTR | 4 | 2375 | gggaaggagaaccctagtcc | 76 | 170 | 370 |
| 134417 | 3′UTR | 4 | 2590 | caggacgacagcttcagtgt | 85 | 171 | 370 |
| 344174 | 3′UTR | 4 | 3119 | gaaacctaccaacagttcct | 82 | 172 | 370 |

As shown in Table 1, SEQ ID NOs 29, 31, 32, 33, 34, 37, 38, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 72, 73, 74, 75, 76, 77, 78, 79, 80, 81, 82, 83, 85, 86, 90, 93, 95, 96, 97, 98, 99, 101, 105, 106, 110, 111, 112, 113, 114, 115, 116, 117, 118, 119, 120, 121, 122, 123, 124, 125, 126, 127, 128, 129, 130, 131, 132, 133, 134, 135, 136, 138, 140, 141, 142, 143, 144, 146, 147, 148, 149, 150, 151, 152, 153, 154, 155, 156, 159, 160, 161, 162, 163, 165, 166, 167, 168, 169, 170, 171 and 172 demonstrated at least 23% inhibition of human DC-SIGN expression in this assay and are therefore preferred. More preferred are SEQ ID NOs 66, 98, 114 and 83. The target regions to which these preferred sequences are complementary are herein referred to as “preferred target segments” and are therefore preferred for targeting by compounds of the present invention. These preferred target segments are shown in Table 3. These sequences are shown to contain thymine (T) but one of skill in the art will appreciate that thymine (T) is generally replaced by uracil (U) in RNA sequences. The sequences represent the reverse complement of the preferred antisense compounds shown in Table 1. “Target site” indicates the first (5′-most) nucleotide number on the particular target nucleic acid to which the oligonucleotide binds. Also shown in Table 3 is the species in which each of the preferred target segments was found.

### Example 16

Antisense Inhibition of Mouse DC-SIGN Expression by Chimeric Phosphorothioate Oligonucleotides Having 2′-MOE Wings and a Deoxy Gap.

In accordance with the present invention, a second series of antisense compounds was designed to target different regions of the mouse DC-SIGN RNA, using published sequences (GenBank accession number XM_{—}134025.1, incorporated herein as SEQ ID NO: 11). The compounds are shown in Table 2. “Target site” indicates the first (5′-most) nucleotide number on the particular target nucleic acid to which the compound binds. All compounds in Table 2 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on mouse DC-SIGN mRNA levels by quantitative real-time PCR as described in other examples herein. Data are from an experiment in which dendritic cells differentiated from bone marrow were treated with 10 nM of the antisense oligonucleotides of the present invention. The positive control for each datapoint is identified in the table by sequence ID number. If present, “N.D.” indicates “no data”.

TABLE 2

Inhibition of mouse DC-SIGN mRNA levels by chimeric
phosphorothioat oligonucleotides having 2′-MOE wings
and a deoxy gap

| | | | | | | |
|---|---|---|---|---|---|---|
| | | TARGET | | | | |
| | | SEQ ID | TARGET | | % | SEQ ID |
| ISIS # | REGION | NO | SITE | SEQUENCE | INHIB | NO |

| | | | | | | |
|---|---|---|---|---|---|---|
| 290107 | 5′UTR | 11 | 1 | cctccttgaatcttacttag | 29 | 173 |
| 290108 | 5′UTR | 11 | 31 | tctgacagagactacagctt | 14 | 174 |
| 290109 | 5′UTR | 11 | 46 | ttctcagtgccagaatctga | 62 | 175 |
| 290110 | Start | 11 | 60 | tgtttcacagccacttctca | 66 | 176 |
| | Codon | | | | | |
| 290111 | Coding | 11 | 87 | tcttccccatttccttagaa | 31 | 177 |
| 290112 | Coding | 11 | 122 | tgtcagcagttcctcatcca | 80 | 178 |
| 290113 | Coding | 11 | 147 | tgatggagtgcctggtgtgg | 69 | 179 |
| 290114 | Coding | 11 | 172 | gaatttgtttggaagccaaa | 57 | 180 |
| 290115 | Coding | 11 | 201 | ccaggcaccctgtgaagcta | 41 | 181 |
| 290116 | Coding | 11 | 241 | aggaagagcacctgcagtgc | 81 | 182 |
| 290117 | Coding | 11 | 278 | gacaaggatgacaaccagca | 65 | 183 |
| 290118 | Coding | 11 | 339 | gttcttggtagacattcatc | 53 | 184 |
| 290119 | Coding | 11 | 358 | ccagccttcaactgggtcag | 48 | 185 |
| 290120 | Coding | 11 | 381 | aggagcggcacagtcgatct | 65 | 186 |
| 290121 | Coding | 11 | 420 | agtaacagcttccttggaag | 50 | 187 |
| 290122 | Coding | 11 | 459 | tggcagaatcattccaggac | 75 | 188 |
| 290123 | Coding | 11 | 501 | tcttgatgaccacaagttga | 44 | 189 |
| 290124 | Coding | 11 | 552 | tgtagcctctcttcttagaa | 66 | 190 |
| 290125 | Coding | 11 | 592 | catgtagactccttgctcat | 52 | 191 |
| 290126 | Coding | 11 | 665 | caggttgttaggttctcctt | 75 | 192 |
| 290127 | Coding | 11 | 700 | ccgtcatctctgaactctgc | 84 | 193 |
| 290128 | Coding | 11 | 749 | ctttttgcagatccagaatt | 59 | 194 |
| 290129 | Stop | 11 | 786 | gagttggccatcacttgcta | 80 | 195 |
| | Codon | | | | | |
| 290130 | Stop | 11 | 795 | tggtggagggagttggccat | 30 | 196 |
| | Codon | | | | | |
| 290131 | 3′UTR | 11 | 852 | actggcatagctatgggtaa | 78 | 197 |
| 290132 | 3′UTR | 11 | 891 | cttgtcaaggttatcaatgg | 65 | 198 |
| 290133 | 3′UTR | 11 | 932 | tgcaggagacaagctacctg | 65 | 199 |
| 290134 | 3′UTR | 11 | 970 | cattaatggtccaggagaca | 31 | 200 |
| 290135 | 3′UTR | 11 | 1010 | cactaacccaaagagaacct | 80 | 201 |
| 290136 | 3′UTR | 11 | 1057 | atgtctgtttacttcattaa | 76 | 202 |
| 290137 | 3′UTR | 11 | 1100 | aggaagacagaggagatgaa | 44 | 203 |
| 290138 | 3′UTR | 11 | 1137 | tcaattgcacatggcaatat | 35 | 204 |
| 290139 | 3′UTR | 11 | 1187 | aggtgtgcagggtagatgaa | 73 | 205 |
| 290140 | 3′UTR | 11 | 1216 | tcctgaaatcagagaagggt | 67 | 206 |
| 290141 | 3′UTR | 11 | 1234 | ttagtctataggttgagctc | 69 | 207 |
| 290142 | 3′UTR | 11 | 1268 | gactaatagttaaggaattg | 62 | 208 |
| 290143 | 3′UTR | 11 | 1303 | ggagtcagagaattaacaca | 60 | 209 |
| 290144 | 3′UTR | 11 | 1333 | ggctcaccatgtccacccag | 82 | 210 |
| 290145 | 3′UTR | 11 | 1397 | ttaatgagtaagttcaataa | 40 | 211 |
| 290146 | 3′UTR | 11 | 1430 | acttatatttatacatacat | 26 | 212 |

As shown in Table 2, SEQ ID NOs 173, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211 and 212 demonstrated at least 20% inhibition of mouse DC-SIGN expression. The target regions to which these sequences are complementary are herein referred to as “target segments” and are shown in Table 3. These sequences are shown to contain thymine (T) but one of skill in the art will appreciate that thymine (T) is generally replaced by uracil (U) in RNA sequences. The sequences represent the reverse complement of the preferred antisense compounds shown in Tables 1 and 2. “Target site” indicates the first (5′-most) nucleotide number on the particular target nucleic acid to which the oligonucleotide binds. Also shown in Table 3 is the species in which each of the preferred target segments was found.

TABLE 3

Sequence and position target segments identified in DC-SIGN

| | | | | | | |
|---|---|---|---|---|---|---|
| | TARGET | | | REV | | |
| | SEQ ID | TARGET | | COMP OF | | SEQ ID |
| SITE ID | NO | SITE | SEQUENCE | SEQ ID | ACTIVE IN | NO |

| | | | | | | |
|---|---|---|---|---|---|---|
| 257999 | 4 | 1 | tggggtgacatgagtgactc | 29 | H. sapiens | 213 |
| 258001 | 4 | 77 | ttggattccgacagactcga | 31 | H. sapiens | 214 |
| 258002 | 4 | 100 | tacaagagcttagcagggtg | 32 | H. sapiens | 215 |
| 258003 | 4 | 166 | ttggctgggctccttgtcca | 33 | H. sapiens | 216 |
| 258004 | 4 | 486 | gcttccagagaaatctaaga | 34 | H. sapiens | 217 |
| 258007 | 4 | 1117 | aatcttgccaaattctggat | 37 | H. sapiens | 218 |
| 258008 | 4 | 1240 | ttttaagctacagttccttc | 38 | H. sapiens | 219 |
| 258011 | 4 | 1316 | tcctttagaaggctgggtcc | 41 | H. sapiens | 220 |
| 258012 | 4 | 1441 | ggtggacttgtgttccaaga | 42 | H. sapiens | 221 |
| 258013 | 4 | 1485 | attaggatatttgggttgcc | 43 | H. sapiens | 222 |
| 258014 | 4 | 1590 | ccctttgatcggggacttac | 44 | H. sapiens | 223 |
| 258015 | 4 | 2176 | acgactttgggatctagagg | 45 | H. sapiens | 224 |
| 258016 | 4 | 2185 | ggatctagaggagctttggc | 46 | H. sapiens | 225 |
| 258017 | 4 | 2201 | tggctccggctgtgacggct | 47 | H. sapiens | 226 |
| 258018 | 4 | 2224 | gccgttctcactgcggctgc | 48 | H. sapiens | 227 |
| 258019 | 4 | 2291 | tgcctctcctggctctttga | 49 | H. sapiens | 228 |
| 258020 | 4 | 2369 | tggtctggactagggttctc | 50 | H. sapiens | 229 |
| 258021 | 4 | 2395 | cgcagcctgtagtattcatc | 51 | H. sapiens | 230 |
| 258022 | 4 | 2405 | agtattcatcgtggtggccc | 52 | H. sapiens | 231 |
| 258023 | 4 | 2459 | aggagacaagcagagqtctc | 53 | H. sapiens | 232 |
| 258024 | 4 | 2473 | ggtctctattggatttctca | 54 | H. sapiens | 233 |
| 258025 | 4 | 2485 | atttctcaacacctgaagaa | 55 | H. sapiens | 234 |
| 258026 | 4 | 2531 | tgttgtatttcaaataaacc | 56 | H. sapiens | 235 |
| 258027 | 4 | 2580 | ggcagaattcacactgaagc | 57 | H. sapiens | 236 |
| 258028 | 4 | 2629 | taaacagagaaagagctgct | 58 | H. sapiens | 237 |
| 258029 | 4 | 2664 | gactggtactgaacatatat | 59 | H. sapiens | 238 |
| 258030 | 4 | 2709 | gttgcaactgaccttgaact | 60 | H. sapiens | 239 |
| 258031 | 4 | 2720 | ccttgaactgaatcactctc | 61 | H. sapiens | 240 |
| 258032 | 4 | 3077 | cccacgttcttgaactcggg | 62 | H. sapiens | 241 |
| 258033 | 4 | 3093 | cgggcagcacatcctcacag | 63 | H. sapiens | 242 |
| 258034 | 4 | 3108 | cacagaaatctaggaactgt | 64 | H. sapiens | 243 |
| 258035 | 4 | 3170 | tcatagtcatccctcctgca | 65 | H. sapiens | 244 |
| 258036 | 4 | 3202 | aaacactgtaaacatgcttt | 66 | H. sapiens | 245 |
| 258037 | 4 | 3213 | acatgcttttaataagaagg | 67 | H. sapiens | 246 |
| 258038 | 4 | 3246 | ttgggaaatcatgtgaacat | 68 | H. sapiens | 247 |
| 258039 | 4 | 3268 | atctccaaatctgcaagctc | 69 | H. sapiens | 248 |
| 258040 | 4 | 3283 | agctcctgttttactgtaga | 70 | H. sapiens | 249 |
| 258042 | 4 | 3664 | cctcctctttctgtgagagt | 72 | H. sapiens | 250 |
| 258043 | 4 | 3681 | agtcctgagtccctcagtgg | 73 | H. sapiens | 251 |
| 258044 | 4 | 3731 | ctccttcttcagggcggccc | 74 | H. sapiens | 252 |
| 258045 | 4 | 3787 | ccttttcgggcgcagccatc | 75 | H. sapiens | 253 |
| 258046 | 4 | 3794 | gggcgcagccatcttgccgg | 76 | H. sapiens | 254 |
| 258047 | 4 | 3863 | tgtaaccgcagattcagccc | 77 | H. sapiens | 255 |
| 258048 | 4 | 3974 | agatgaacgaagcgtgcaaa | 78 | H. sapiens | 256 |
| 258049 | 4 | 4008 | aagtgctcagactgaggaac | 79 | H. sapiens | 257 |
| 258050 | 4 | 4017 | gactgaggaacagggactga | 80 | H. sapiens | 258 |
| 258051 | 4 | 4028 | agggactgagttaagaagtg | 81 | H. sapiens | 259 |
| 258052 | 4 | 4090 | tggcgtgatccagtcagatc | 82 | H. sapiens | 260 |
| 258053 | 4 | 4101 | agtcagatcaagcctcctga | 83 | H. sapiens | 261 |
| 258059 | 20 | 458 | tgaaggctgcagtggaacgc | 85 | H. sapiens | 262 |
| 258060 | 21 | 200 | taagtcaggaacaatccaga | 86 | H. sapiens | 263 |
| 258064 | 25 | 919 | tgaggagcagcttcaagcag | 90 | H. sapiens | 264 |
| 258067 | 27 | 632 | agcttagcaggtagagagga | 93 | H. sapiens | 265 |
| 258069 | 27 | 1882 | aagtcaggaacaatccaggc | 95 | H. sapiens | 266 |
| 258070 | 27 | 2016 | gtgagcttccagagaaatct | 96 | H. sapiens | 267 |
| 258071 | 27 | 2109 | tgcaggagatctaccaggag | 97 | H. sapiens | 268 |
| 258072 | 27 | 2144 | gctgcagtgggtgagcttcc | 98 | H. sapiens | 269 |
| 258073 | 27 | 2997 | tgaggagcaggtacacctgg | 99 | H. sapiens | 270 |
| 258075 | 27 | 3736 | acagctgcagtcttccagaa | 101 | H. sapiens | 271 |
| 258079 | 4 | 134 | ccctggtgctgcaactcctc | 105 | H. sapiens | 272 |
| 258080 | 4 | 139 | gtgctgcaactcctctcctt | 106 | H. sapiens | 273 |
| 258084 | 4 | 171 | tgggctccttgtccaagtgt | 110 | H. sapiens | 274 |
| 258085 | 4 | 275 | gtgagctctcagagaaatcc | 111 | H. sapiens | 275 |
| 258086 | 4 | 284 | cagagaaatccaagctgcag | 112 | H. sapiens | 276 |
| 258087 | 4 | 289 | aaatccaagctgcaggagat | 113 | H. sapiens | 277 |
| 258088 | 4 | 294 | caagctgcaggagatctacc | 114 | H. sapiens | 278 |
| 258089 | 4 | 304 | gagatctaccaggagctgac | 115 | H. sapiens | 279 |
| 258090 | 4 | 317 | agctgacccagctgaaggct | 116 | H. sapiens | 280 |
| 258091 | 4 | 322 | acccagctgaaggctgcagt | 117 | H. sapiens | 281 |
| 258092 | 4 | 328 | ctgaaggctgcagtgggtga | 118 | H. sapiens | 282 |
| 258093 | 4 | 333 | ggctgcagtgggtgagcttc | 119 | H. sapiens | 283 |
| 258094 | 4 | 338 | cagtgggtgagcttccagag | 120 | H. sapiens | 284 |
| 258095 | 4 | 343 | ggtgagcttccagagaaatc | 121 | H. sapiens | 285 |
| 258096 | 4 | 364 | aagctgcaggagatctacca | 122 | H. sapiens | 286 |
| 258097 | 4 | 369 | gcaggagatctaccaggagc | 123 | H. sapiens | 287 |
| 258098 | 4 | 374 | agatctaccaggagctgacc | 124 | H. sapiens | 288 |
| 258099 | 4 | 379 | taccaggagctgacccggct | 125 | H. sapiens | 289 |
| 258100 | 4 | 384 | ggagctgacccggctgaagg | 126 | H. sapiens | 290 |
| 258101 | 4 | 389 | tgacccggctgaaggctgca | 127 | H. sapiens | 291 |
| 258102 | 4 | 448 | taccaggagctgacctggct | 128 | H. sapiens | 292 |
| 258103 | 4 | 453 | ggagctgacctggctgaagg | 129 | H. sapiens | 293 |
| 258104 | 4 | 458 | tgacctggctgaaggctgca | 130 | H. sapiens | 294 |
| 258105 | 4 | 491 | cagagaaatctaagatgcag | 131 | H. sapiens | 295 |
| 258106 | 4 | 496 | aaatctaagatgcaggagat | 132 | H. sapiens | 296 |
| 258107 | 4 | 501 | taagatgcaggagatctacc | 133 | H. sapiens | 297 |
| 258108 | 4 | 571 | aagcagcaggagatctacca | 134 | H. sapiens | 298 |
| 258109 | 4 | 741 | gctgaaggctgcagtggaac | 135 | H. sapiens | 299 |
| 258110 | 4 | 787 | tggacattcttccaaggaaa | 136 | H. sapiens | 300 |
| 258112 | 4 | 814 | ttcatgtctaactcccagcg | 138 | H. sapiens | 301 |
| 258114 | 4 | 824 | actcccagcggaactggcac | 140 | H. sapiens | 302 |
| 258115 | 4 | 829 | cagcggaactggcacgactc | 141 | H. sapiens | 303 |
| 258116 | 4 | 834 | gaactggcacgactccatca | 142 | H. sapiens | 304 |
| 258117 | 4 | 868 | gtgggggcccagctcgtcgt | 143 | H. sapiens | 305 |
| 258118 | 4 | 873 | ggcccagctcgtcgtaatca | 144 | H. sapiens | 306 |
| 258120 | 4 | 883 | gtcgtaatcaaaagtgctga | 146 | H. sapiens | 307 |
| 258121 | 4 | 888 | aatcaaaagtgctgaggagc | 147 | H. sapiens | 308 |
| 258122 | 4 | 893 | aaagtgctgaggagcagaac | 148 | H. sapiens | 309 |
| 258123 | 4 | 898 | gctgaggagcagaacttcct | 149 | H. sapiens | 310 |
| 258124 | 4 | 919 | cagctgcagtcttccagaag | 150 | H. sapiens | 311 |
| 258125 | 4 | 924 | gcagtcttccagaagtaacc | 151 | H. sapiens | 312 |
| 258126 | 4 | 929 | cttccagaagtaaccgcttc | 152 | H. sapiens | 313 |
| 258127 | 4 | 934 | agaagtaaccgcttcacctg | 153 | H. sapiens | 314 |
| 258128 | 4 | 939 | taaccgcttcacctggatgg | 154 | H. sapiens | 315 |
| 258129 | 4 | 944 | gcttcacctggatgggactt | 155 | H. sapiens | 316 |
| 258130 | 4 | 949 | acctggatgggactttcaga | 156 | H. sapiens | 317 |
| 258133 | 4 | 1013 | tgttgcccagcttcaagcag | 159 | H. sapiens | 318 |
| 258134 | 4 | 1018 | cccagcttcaagcagtattg | 160 | H. sapiens | 319 |
| 258135 | 4 | 1023 | cttcaagcagtattggaaca | 161 | H. sapiens | 320 |
| 258136 | 4 | 1078 | gcggaatttagtggcaatgg | 162 | H. sapiens | 321 |
| 258137 | 4 | 1083 | atttagtggcaatggctgga | 163 | H. sapiens | 322 |
| 258139 | 4 | 1269 | ttcgaccttcacaaaatctc | 165 | H. sapiens | 323 |
| 258140 | 4 | 1329 | tgggtcccattctgtccttc | 166 | H. sapiens | 324 |
| 258141 | 4 | 1377 | agagcttgtttttctggccc | 167 | H. sapiens | 325 |
| 258142 | 4 | 1474 | tccttttggagattaggata | 168 | H. sapiens | 326 |
| 258143 | 4 | 2119 | ttactgggatttgggactct | 169 | H. sapiens | 327 |
| 258144 | 4 | 2375 | ggactagggttctccttccc | 170 | H. sapiens | 328 |
| 258145 | 4 | 2590 | acactgaagctgtcgtcctg | 171 | H. sapiens | 329 |
| 258146 | 4 | 3119 | aggaactgttggtaggtttc | 172 | H. sapiens | 330 |
| 206084 | 11 | 1 | ctaagtaagattcaaggagg | 173 | M. musculus | 331 |
| 206086 | 11 | 46 | tcagattctggcactgagaa | 175 | M. musculus | 332 |
| 206087 | 11 | 60 | tgagaagtggctgtgaaaca | 176 | M. musculus | 333 |
| 206088 | 11 | 87 | ttctaaggaaatggggaaga | 177 | M. musculus | 334 |
| 206089 | 11 | 122 | tggatgaggaactgctgaca | 178 | M. musculus | 335 |
| 206090 | 11 | 147 | ccacaccaggcactccatca | 179 | M. musculus | 336 |
| 206091 | 11 | 172 | tttggcttccaaacaaattc | 180 | M. musculus | 337 |
| 206092 | 11 | 201 | tagcttcacagggtgcctgg | 181 | M. musculus | 338 |
| 206093 | 11 | 241 | gcactgcaggtgctcttcct | 182 | M. musculus | 339 |
| 206094 | 11 | 278 | tgctggttgtcatccttgtc | 183 | M. musculus | 340 |
| 206095 | 11 | 339 | gatgaatgtctaccaagaac | 184 | M. musculus | 341 |
| 206096 | 11 | 358 | ctgacccagttgaaggctgg | 185 | M. musculus | 342 |
| 206097 | 11 | 381 | agatcgactgtgccgctcct | 186 | M. musculus | 343 |
| 206098 | 11 | 420 | cttccaaggaagctgttact | 187 | M. musculus | 344 |
| 206099 | 11 | 459 | gtcctggaatgattctgcca | 188 | M. musculus | 345 |
| 206100 | 11 | 501 | tcaacttgtggtcatcaaga | 189 | M. musculus | 346 |
| 206101 | 11 | 552 | ttctaagaagagaggctaca | 190 | M. musculus | 347 |
| 206102 | 11 | 592 | atgagcaaggagtctacatg | 191 | M. musculus | 348 |
| 206103 | 11 | 665 | aaggagaacctaacaacctg | 192 | M. musculus | 349 |
| 206104 | 11 | 700 | gcagagttcagagatgacgg | 193 | M. musculus | 350 |
| 206105 | 11 | 749 | aattctggatctgcaaaaag | 194 | M. musculus | 351 |
| 206106 | 11 | 786 | tagcaagtgatggccaactc | 195 | M. musculus | 352 |
| 206107 | 11 | 795 | atggccaactccctccacca | 196 | M. musculus | 353 |
| 206108 | 11 | 852 | ttacccatagctatgccagt | 197 | M. musculus | 354 |
| 206109 | 11 | 891 | ccattgataaccttgacaag | 198 | M. musculus | 355 |
| 206110 | 11 | 932 | caggtagcttgtctcctgca | 199 | M. musculus | 356 |
| 206111 | 11 | 970 | tgtctcctggaccattaatg | 200 | M. musculus | 357 |
| 206112 | 11 | 1010 | aggttctctttgggttagtg | 201 | M. musculus | 358 |
| 206113 | 11 | 1057 | ttaatgaagtaaacagacat | 202 | M. musculus | 359 |
| 206114 | 11 | 1100 | ttcatctcctctgtcttcct | 203 | M. musculus | 360 |
| 206115 | 11 | 1137 | atattgccatgtgcaattga | 204 | M. musculus | 361 |
| 206116 | 11 | 1187 | ttcatctaccctgcacacct | 205 | M. musculus | 362 |
| 206117 | 11 | 1216 | acccttctctgatttcagga | 206 | M. musculus | 363 |
| 206118 | 11 | 1234 | gagctcaacctatagactaa | 207 | M. musculus | 364 |
| 206119 | 11 | 1268 | caattccttaactattagtc | 208 | M. musculus | 365 |
| 206120 | 11 | 1303 | tgtgttaattctctgactcc | 209 | M. musculus | 366 |
| 206121 | 11 | 1333 | ctgggtggacatggtgagcc | 210 | M. musculus | 367 |
| 206122 | 11 | 1397 | ttattgaacttactcattaa | 211 | M. musculus | 368 |
| 206123 | 11 | 1430 | atgtatgtataaatataagt | 212 | M. musculus | 369 |

As these “target segments” have been found by experimentation to be open to, and accessible for, hybridization with the antisense compounds of the present invention, one of skill in the art will recognize or be able to ascertain, using no more than routine experimentation, further embodiments that encompass other compounds that specifically hybridize to these target segments and consequently inhibit the expression of DC-SIGN.

According to the present invention, antisense compounds include antisense oligomeric compounds, antisense oligonucleotides, ribozymes, external guide sequence (EGS) oligonucleotides, alternate splicers, primers, probes, and other short oligomeric compounds which hybridize to at least a portion of the target nucleic acid.

### Example 17

Western Blot Analysis of DC-SIGN Protein Levels

Western blot analysis (immunoblot analysis) is carried out using standard methods. Cells are harvested 16-20 h after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to DC-SIGN is used, with a radiolabeled or fluorescently labeled secondary antibody directed against the primary antibody species. Bands are visualized using a PHOSPHORIMAGER™ (Molecular Dynamics, Sunnyvale Calif.).

### Example 18

Mouse Model of Allergic Inflammation

In the mouse model of allergic inflammation, mice were sensitized and challenged with aerosolized chicken ovalbumin (OVA). Airway responsiveness was assessed by inducing airflow obstruction with a methacholine aerosol using a noninvasive method. This methodology utilized unrestrained conscious mice that are placed into the main chamber of a plthysmograph (Buxco Electronics, Inc., Troy, N.Y.). Pressure differences between this chamber and a reference chamber were used to extrapolate minute volume, breathing frequency and enhanced pause (Penh). Penh is a dimensionless parameter that is a function of total pulmonary airflow in mice (i.e., the sum of the airflow in the upper and lower respiratory tracts) during the respiratory cycle of the animal. The lower the Penh, the greater the airflow. This parameter closely correlates with lung resistance as measured by traditional invasive techniques using ventilated animals (Hamelmann . . . Gelfand, 1997). Dose-response data were plotted as raw Penh values to increasing concentrations of methacholine. This system was used to test the efficacy of antisense oligonucleotides targeted to DC-SIGN.

There are several important features common to human asthma and the mouse model of allergic inflammation. One of these is pulmonary inflammation, in which cytokine expression and Th2 profile is dominant. Another is goblet cell hyperplasia with increased mucus production. Lastly, airway hyperresponsiveness (AHR) occurs resulting in increased sensitivity to cholinergic receptor agonists such as acetylcholine or methacholine. The compositions and methods of the present invention may be used to treat AHR and pulmonary inflammation.

Ovalbumin-induced Allergic Inflammation

Balb/c mice (Charles Rivers Laboratory, Taconic Farms, NY), 8-10 weeks of age, weighing about 25 g each, were maintained in micro-isolator cages housed in a specific pathogen-free (SPF) facility. The sentinel cages within the animal colony surveyed negative for viral antibodies and the presence of known mouse pathogens. Mice were sensitized and challenged with aerosolized chicken OVA. Briefly, 20 μg alum-precipitated OVA was injected intraperitoneally on days 0 and 14. On day 24, 25 and 26, the animals were exposed for 20 minutes to 1.0% OVA (in saline) by nebulization. The challenge was conducted using an ultrasonic nebulizer (PulmoSonic, The DeVilbiss Co., Somerset, Pa.). Animals were analyzed about 24 hours following the last nebulization using the Buxco electronics Biosystem. Lung function (Penh), lung histology (cell infiltration and mucus production) inflammation (BAL cell type & number) and spleen weight were determined.

Intratracheal Oligonucleotide Administration

Antisense oligonucleotides (ASOs) were dissolved in saline and used to intratracheally dose mice every day, four times per day, from days 15-26 of the OVA sensitization and challenge protocol. There were 5 mice/group for day 25 (cytokine measurements) and 10 mice/group for Penh and broncheoalveolar (BAL) fluid cell type analysis. The only group not sensitized with OVA was naive mice. Specifically, the mice were anesthetized with isofluorane and placed on a board with the front teeth hung from a line. The nose was covered and the animal's tongue was extended with forceps and 25 μl of various doses of ASO, or an equivalent volume of saline (control) was placed at the back of the tongue until inhaled into the lung. On day 28, lung function measurements (Penh) were taken. The DC-SIGN oligonucleotides used were ISIS 290127 and 290135. No cross hybridization was predicted, and no cross-target reduction was detected in transfected cells. The treatment groups are shown in Table 4:

TABLE 4

| | | Dose | | |
|---|---|---|---|---|
| Groups | Treatment | (mg/kg) | Day 25 sac | Day 28 sac |
| 1. (N = 15) | ISIS | 0.001 | N = 5 | N = 10 |
| | 290127 | | | |
| 2. (N = 15) | ISIS | 0.01 | N = 5 | N = 10 |
| | 290127 | | | |
| 3. (N = 15) | ISIS | 0.1 | N = 5 | N = 10 |
| | 290127 | | | |
| 4. (N = 15) | 290135 | 0.001 | N = 5 | N = 10 |
| 5. (N = 15) | ISIS | 0.01 | N = 5 | N = 10 |
| | 290135 | | | |
| 6. (N = 15) | ISIS | 0.1 | N = 5 | N = 10 |
| | 290135 | | | |
| 7. (N = 15) | ISIS | 0 | N = 5 | N = 10 |
| | 290135 | | | |
| 8. (N = 15) | Naive | 0 | N = 5 | N = 10 |

### Example 19

Aerosol Administration of DC-SIGN Antisense Oligonucleotides

In the aerosolized DC-SIGN oligonucleotide study, treatment with 0.0003, 0.0003 or 0.3 μg/kg estimated inhaled dose was delivered by nose-only inhalation of an aerosol solution of ISIS 290129, ISIS 290135 or vehicle, four times per day, on days 15-26 (n=8 mice per group). The airway response to methacholine was reduced to the level seen in naive mice at 0.001 mg/kg dose (estimated inhaled dose=1 μg/kg). No gross adverse effects were seen. All groups were sensitized with OVA except naive mice. The treatment groups are shown in Table

TABLE 5

| | | | Day 25 or | |
|---|---|---|---|---|
| | | | 26 for BAL | |
| Groups | Treatment | Dose | cytokines | Day 28 sac |
| 1. (N = 15) | ISIS | 0.00003 | N = 5 | N = 10 |
| | 290129 | | | |
| 2. (N = 15) | ISIS | 0.003 | N = 5 | N = 10 |
| | 290129 | | | |
| 3. (N = 15) | ISIS | 0.3 | N = 5 | N = 10 |
| | 290129 | | | |
| 4. (N = 15) | ISIS | 0.00003 | N = 5 | N = 10 |
| | 290135 | | | |
| 5. (N = 15) | ISIS | 0.003 | N = 5 | N = 10 |
| | 290135 | | | |
| 6. (N = 15) | ISIS | 0 | N = 5 | N = 10 |
| | 290135 | | | |
| 7. (N = 15) | vehicle | 0 | N = 5 | N = 10 |
| 8. (N = 15) | Naive | 0 | N = 5 | N = 10 |

### Example 20

Collection of Bronchial Alveolar Lavage (BAL) Fluid and Blood Serum for the Determination of Cytokine and Chemokine Levels

Animals were injected with a lethal dose of ketamine, the trachea was exposed and a cannula was inserted and secured by sutures. The lungs were lavaged twice with 0.5 ml aliquots of ice cold PBS with 0.2% FCS. The recovered BAL fluid was centrifuged at 1,000 rpm for 10 min at 4° C., frozen on dry ice and stored at −80° C. until used. Luminex was used to measure cytokine levels in BAL fluid and serum.

### Example 21

BAL Cell Counts and Differentials

Cytospins of cells recovered from BAL fluid were prepared using a Shandon Cytospin 3 (Shandon Scientific LTD, Cheshire, England). Cell differentials were performed from slides stained with Leukostat (Fisher Scientific, Pittsburgh, Pa.). Total cell counts were quantified by hemocytometer and, together with the percent type bty differential, were used to calculate specific cell number.

### Example 22

Mucus Cell Content

Mucus cell content was assessed as the airway epithelium staining with periodic acid-schiff (PAS) reagent. Relative comparisons of mucus content were made between cohorts of animals by counting the number of PAS-positive airways.

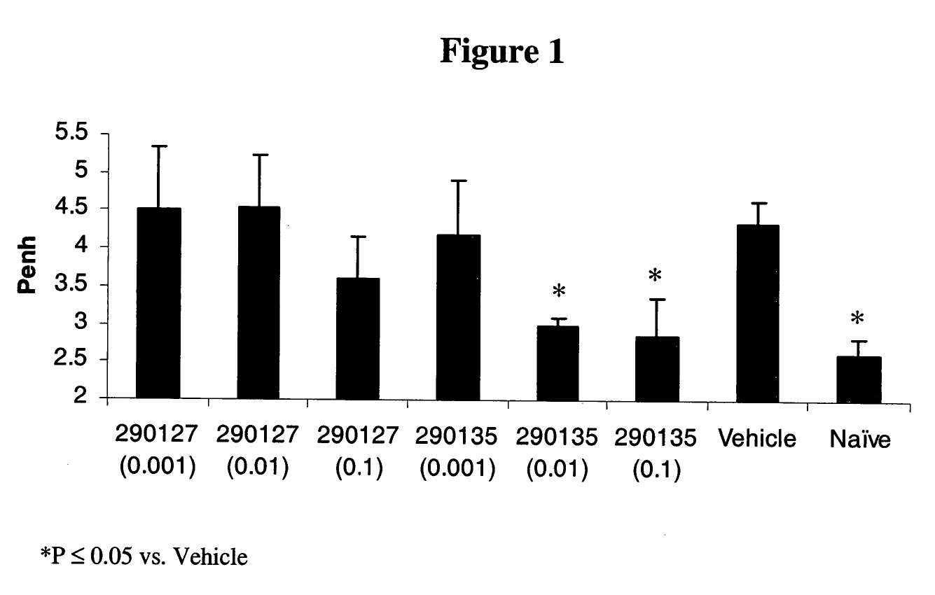

The results show a pronounced decrease in Penh after administration of each oligonucleotide which translates to decreased airway hyperresponsiveness in mice after intratracheal administration (FIG. 1). Neither oligonucleotide resulted in increased spleen weight, thus they were not toxic.

Intratracheal Administration Results

As shown in FIG. 1, treatment with ISIS 290127 or ISIS 290135 following allergen (OVA) challenge in the mouse model of asthma reduces the airway response to methacholine (MCH, 100 mg/ml), with ISIS 290135 showing a more pronounced effect. The Penh value in ISIS 290135-treated mice was about one-third lower than vehicle-treated mice, and was statistically the same as naive mice which were not sensitized with the allergen or treated with the ASO. This shows that DC-SIGN ASO-treated mice had significantly better airflow, and less inflammation, than mice which were not treated with the ASO.

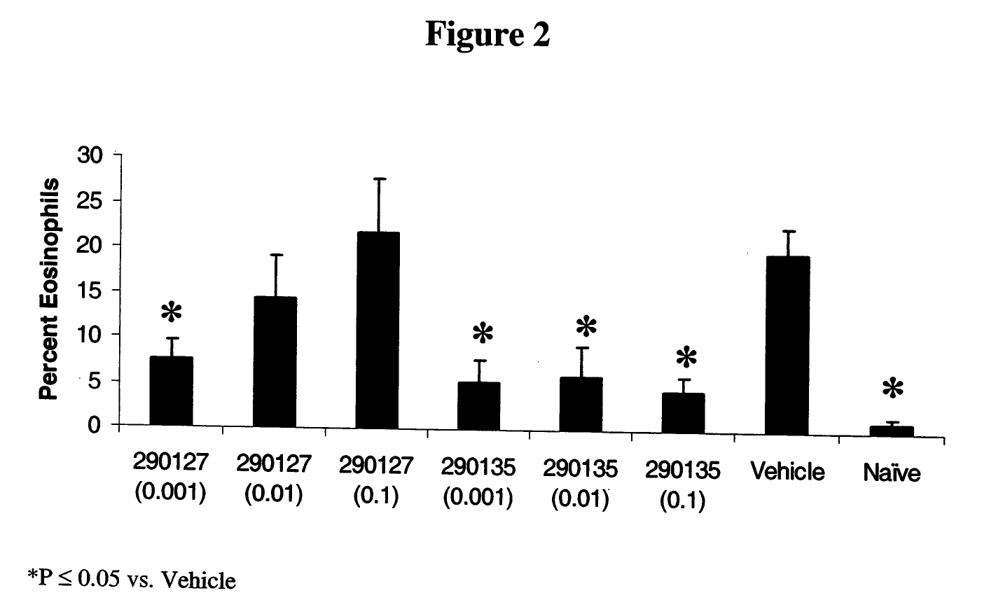

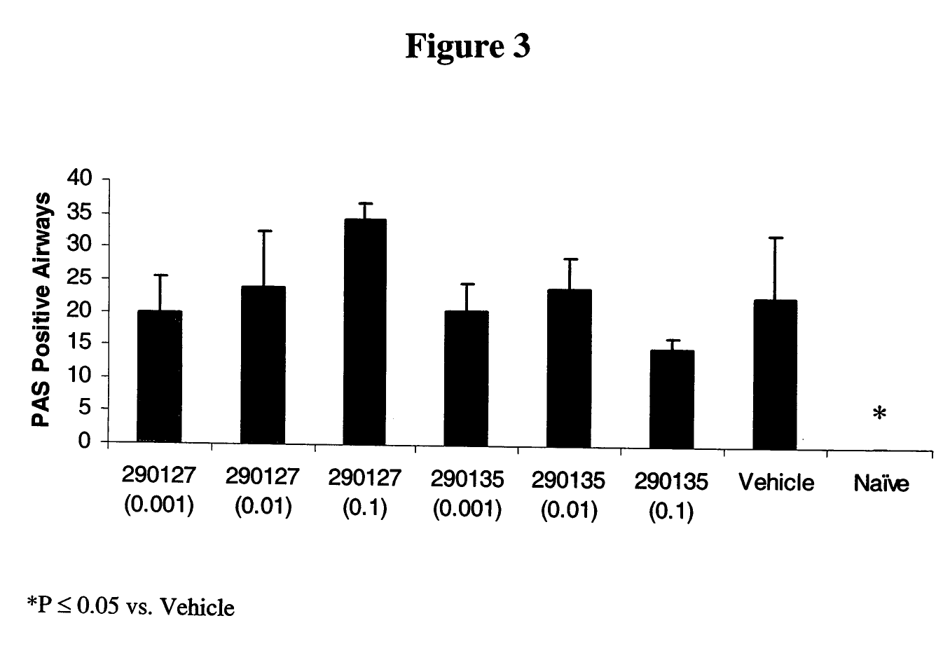

The effect of DC-SIGN antisense oligonucleotides on eosinophil recruitment, as measured from BAL fluid, is shown in FIG. 2. Compared to vehicle treated mice, DC-SIGN ASO treated mice exhibited reduced number of eosinophils, cells which promote the inflammatory response. This was most pronounced with ISIS 290135, but was also seen for ISIS 290127 at the lowest dose. ISIS 290135 at its lowest dose (0.001 mg/kg) reduced the total number of eosinophils by about 65% compared to vehicle-treated mice. Since increased numbers of eosinophils result from inflammation, this provides further support for the anti-inflammatory properties of DC-SIGN antisense oligonucleotides. In addition, these oligonucleotides lower pulmonary mucus production as determined by PAS positive staining of mouse airways (FIG. 3) In addition, daily intratracheal delivery of ISIS 290127 and 290135 do not induce splenomegaly.

In summary, ISIS 290127 and 290135 resulted in an inhibition of airway hypersensitivity, reduced eosinophilia and decreased mucus production in OVA-sensitized mice.

Aerosol Administration Results

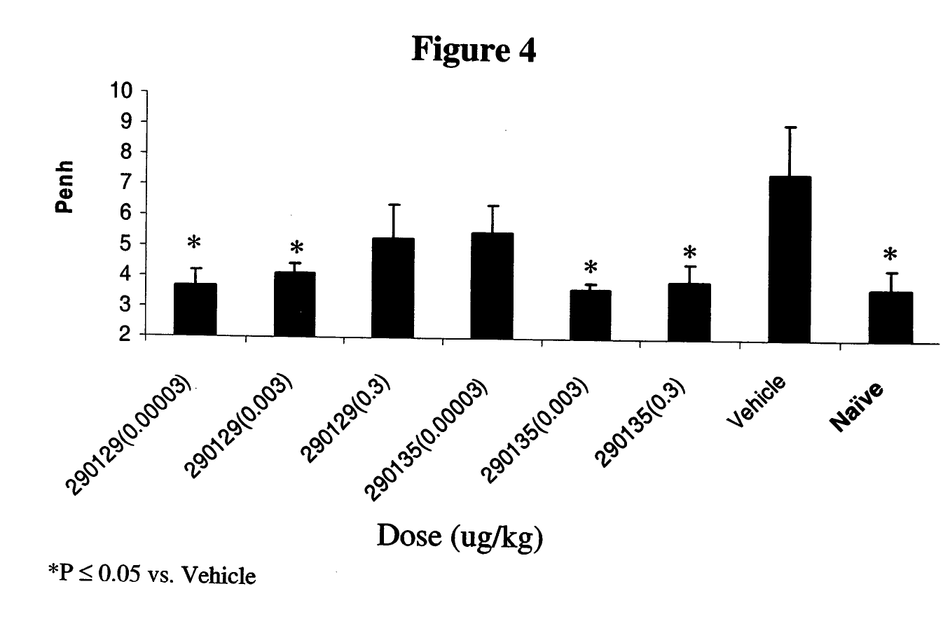

As shown in FIG. 4, treatment with ISIS 290129 or ISIS 290135 following allergen (OVA) challenge in the mouse model of asthma reduces the airway response to methacholine (MCH, 100 mg/ml). The Penh value in mice treated with either oligonucleotide was about 40% lower than vehicle-treated mice, and was statistically the same as naive mice which were not sensitized with the allergen or treated with the ASO. This shows that DC-SIGN ASO-treated mice had significantly better airflow, and less inflammation, than mice which were not treated with the ASO.

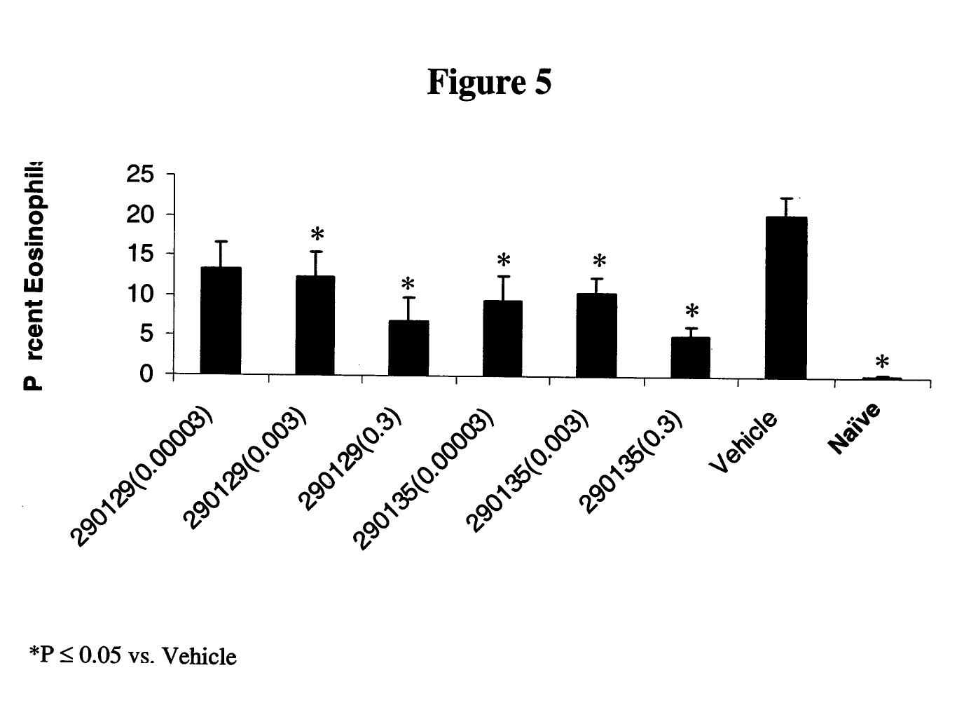

The effect of DC-SIGN antisense oligonucleotides on eosinophil recruitment, as measured from BAL fluid, is shown in FIG. 5. Compared to vehicle treated mice, DC-SIGN ASO treated mice exhibited reduced number of eosinophils, cells which promote the inflammatory response. This was more pronounced with ISIS 290135. ISIS 290135 and ISIS 290129 at their lowest doses (0.003 mg/kg) reduced the total number of eosinophils by about 50% and 35%, respectively, compared to vehicle-treated mice. Since increased numbers of eosinophils result from inflammation, this provides further support for the anti-inflammatory properties of DC-SIGN antisense oligonucleotides.

The combined use of antisense oligonucleotide(s) targeted to DC-SIGN with one or more conventional asthma medications including, but not limited to, montelukast sodium (Singulair™), albuterol, beclomethasone dipropionate, triamcinolone acetonide, ipratropium bromide (Atrovent™), flunisolide, fluticasone propionate (Flovent™) and other steroids is also contemplated.

1. An antisense compound 8 to 80 nucleobases in length targeted to a coding region of a nucleic acid molecule encoding human DC-SIGN (SEQ ID NO: 4), wherein said compound is at least 70% complementary to said nucleic acid molecule encoding DC-SIGN, and wherein said compound inhibits the expression of DC-SIGN mRNA by at least 10%.

2. The antisense compound of claim 1 comprising 12 to 50 nucleobases in length.

3. The antisense compound of claim 2 comprising 15 to 30 nucleobases in length.

4. The antisense compound of claim 1 comprising an oligonucleotide.

5. The antisense compound of claim 4 comprising a DNA oligonucleotide.

6. The antisense compound of claim 4 comprising an RNA oligonucleotide.

7. The antisense compound of claim 4 comprising a chimeric oligonucleotide.

8. The antisense compound of claim 4 wherein at least a portion of said compound hybridizes with RNA to form an oligonucleotide-RNA duplex.

9. The antisense compound of claim 1 having at least 80% complementarity with said nucleic acid molecule encoding DC-SIGN.

10. The antisense compound of claim 1 having at least 90% complementarity with said nucleic acid molecule encoding DC-SIGN.

11. The antisense compound of claim 1 having at least 95% complementarity with said nucleic acid molecule encoding DC-SIGN.

12. The antisense compound of claim 1 having at least 99% complementarity with said nucleic acid molecule encoding DC-SIGN.

13. The antisense compound of claim 1 having at least one modified internucleoside linkage, sugar moiety, or nucleobase.

14. The antisense compound of claim 1 having at least one 2′-O-methoxyethyl sugar moiety.

15. The antisense compound of claim 1 having at least one phosphorothioate internucleoside linkage.

16. The antisense compound of claim 1 wherein at least one cytosine is a 5-methylcytosine.

17. A method of inhibiting the expression of DC-SIGN in a cell or tissue comprising contacting said cell or tissue with the antisense compound of claim 1 so that expression of DC-SIGN is inhibited.

18. A method of treating airway hyperresponsiveness in an animal comprising administering to said animal a therapeutically or prophylactically effective amount of the antisense compound of claim 1 so that expression of DC-SIGN is inhibited.

19. The method of claim 18 wherein said airway hyperresponsiveness is associated with asthma.
